(19) United States
(12) Patent Application Publication
(10) Pub. No.: US 2026/0207643 A1
(43) Pub. Date: Jul. 23, 2026

(54) SIALYLATED SACCHARIDE FOR USE IN THE PREVENTION OR TREATMENT OF AN INFLAMMATORY DISEASE AND/OR AUTOIMMUNE DISEASE

(71) Applicant: INBIOSE N.V., Zwijnaarde (BE)

(72) Inventors: Joeri Beauprez, Zwijnaarde (BE); Ut Van Nguyen, Zwijnaarde (BE); Annelies Vercauteren, Zwijnaarde (BE)

(21) Appl. No.: 19/134,906

(22) PCT Filed: Dec. 5, 2023
(86) PCT No.: PCT/EP2023/084384

§ 371 Date: Jun. 2, 2025

(30) Foreign Application Priority Data
Dec. 5, 2022 (EP) 22211481.1
Dec. 5, 2022 (LU) LU503136

(51) Int. Cl. A61K 31/702; A61K 31/715; A61P 19/02; A61P 37/06

(52) U.S. Cl. CPC A61K 31/702; A61K 31/715; A61P 19/02; A61P 37/06

(57) ABSTRACT

The present invention relates to a sialylated saccharide for use in a method for preventing and/or treating an inflammatory disease and/or autoimmune disease in a subject, more specifically for preventing and/or treating a disease that causes joint inflammation, even more specifically for preventing and/or treating arthritis. The invention further relates to a method for maintaining and/or improving mobility in a healthy subject. Finally, the invention relates to a method for maintaining and/or improving joint function and/or muscle strength in a healthy subject.

### CROSS-REFERENCE TO RELATED APPLICATIONS

This application is a national phase entry under 35 U.S.C. § 371 of International Patent Application PCT/EP2023/084384, filed Dec. 5, 2023, designating the United States of America and published as International Patent Publication WO 2024/121171 A1 on Jun. 13, 2024, which claims the benefit under Article 8 of the Patent Cooperation Treaty of European Patent Application Serial No. 22211481.1, filed Dec. 5, 2022 and the Patent Cooperation Treaty of Luxembourg Patent Application Serial No. LU503136, filed Dec. 5, 2022.

### TECHNICAL FIELD

This disclosure relates to a sialylated saccharide for use in a method for preventing and/or treating an inflammatory disease and/or autoimmune disease in a subject, more specifically for preventing and/or treating a disease that causes joint inflammation, even more specifically for preventing and/or treating arthritis. This disclosure further relates to a method for maintaining and/or improving mobility in a healthy subject. Finally, this disclosure relates to a method for maintaining and/or improving joint function and/or muscle strength in a healthy subject.

### BACKGROUND

Joint health and arthritis constitute one of the most important chronic health issues in humans and animals. The most prevalent type of arthritis is osteoarthritis (OA) and is characterized by chronic degenerative arthropathy that frequently leads to chronic pain and disability. Another prevalent arthritis disease is rheumatoid arthritis and affects the entire body. It is characterized by inflammation of the joint, which causes pain, stiffness, warmth, redness and swelling. This inflammation is a consequence of inflammatory cells invading the joints and these inflammatory cells release enzymes that may digest bone and cartilage. As a result, this inflammation can lead to severe bone and cartilage damage and to joint deterioration and severe pain amongst other physiologic effects. A further example is systemic lupus erythematosus is an autoimmune disease in which the immune system attacks its own tissues, causing widespread inflammation and tissue damage in the affected organs. It can affect joint, skin, brain, lung, kidney and blood vessel. Due to their devastating medical and economic impact, the development of effective disease-modifying therapeutics is imperative.

Furthermore, maintaining or improving joint function and/or muscle strength in a healthy subject is highly needed. Indeed, mobility issues are one of the most cited health problem of ageing individuals and animals, being confronted with joint pain, decrease in muscle strength and/or reduced joint flexibility. Further, one is looking for means to improve joint function and/or to improve muscle strength of athletes and animals involved in sports such as racing horses.

### BRIEF SUMMARY

It was surprisingly found that a sialylated saccharide according to this disclosure proves to be an efficacious agent to prevent and/or treat an inflammatory disease and/or an autoimmune disease in a subject. Furthermore, this sialylated saccharide surprisingly improves mobility in a healthy subject. Furthermore, this sialylated saccharide surprisingly maintains and/or improves joint function and/or muscle strength in a healthy subject. The sialylated saccharide is not associated with adverse effects and is safe to use in humans and animals.

In a first aspect, provided is a sialylated saccharide for use in a method for preventing and/or treating an inflammatory disease and/or autoimmune disease in a subject.

In a second aspect, provided is a method for preventing and/or treating an inflammatory disease and/or an autoimmune disease in a subject, wherein a sialylated saccharide according to the first aspect is administered to the subject.

In a third aspect, provided is the use of a sialylated saccharide according to the first aspect for the manufacture of a medicament for preventing and/or treating an inflammatory disease and/or an autoimmune disease in a subject.

In a fourth aspect, provided is a method to maintain and/or improve mobility in a healthy subject by administering a sialylated saccharide to the subject.

In a fifth aspect, provided is a method to maintain and/or improve joint function and/or muscle strength in a healthy subject by administering a sialylated saccharide to the subject.

### BRIEF DESCRIPTION OF THE DRAWINGS

The following drawings will serve as further illustration and clarification of this disclosure and are not intended to be limiting in any way.

FIG. 1. Changes (in %) of the propulsive force (FYmin) in healthy (H), less lameness (LL), and more lameness (ML) paws as compared to start day (T1) after 2 weeks (T2), 2 months (T3), and 4 months (T4) of treatment with sialyllactose. The Y-axis represents the FYmin change at Tn (i.e., T2, T3 or T4) as compared to T1 ((Tn−T1)/T1). The asterisk indicates significant (P<0.05) difference between sialyllactose treatment and the placebo.

FIG. 2. Changes (in %) of the breaking force (FYmax) in healthy (H), less lameness (LL), and more lameness (ML) paws as compared to start day (T1) after 2 weeks (T2), 2 months (T3), and 4 months (T4) of treatment with sialyllactose. The Y-axis represents the FYmax change at Tn (i.e., T2, T3 or T4) as compared to T1 ((Tn−T1)/T1). The asterisk indicates significant (P<0.01) difference between sialyllactose treatment and the placebo.

FIG. 3. Changes (in %) of the vertical force (FZmax) in healthy (H), less lameness (LL), and more lameness (ML) paws as compared to start day (T1) after 2 weeks (T2), 2 months (T3), and 4 months (T4) of treatment with SLs. The Y-axis represents the FZmax change at Tn (i.e., T2, T3 or T4) as compared to T1 ((Tn−T1)/T1). Each asterisk indicates significant (P<0.05, 0.01, 0.001) difference between sialyllactose treatment and the placebo.

### DETAILED DESCRIPTION

In a first aspect, this disclosure provides a sialylated saccharide for use in a method for preventing and/or treating an inflammatory disease and/or autoimmune disease in a subject.

In the context of this disclosure, the term “preventing” an inflammatory disease and/or autoimmune disease as described herein, preferably means avoiding that the inflammatory disease and/or autoimmune disease occurs and/or decreasing the incidence of the inflammatory disease and/or autoimmune disease. In other words, “preventing” preferably refers to ameliorating the risk of suffering from the inflammatory disease and/or autoimmune disease. The term “treating” an inflammatory disease and/or autoimmune disease as described herein, preferably means inhibiting the inflammatory disease and/or autoimmune disease, e.g., arresting the development of the inflammatory disease and/or autoimmune disease; inhibiting the inflammatory disease and/or autoimmune disease, e.g., arresting the development of the inflammatory disease and/or autoimmune disease; relieving the inflammatory disease and/or autoimmune disease, for example, causing regression of the inflammatory disease and/or autoimmune disease; and/or relieving a condition caused by or resulting from the inflammatory disease and/or autoimmune disease, e.g., relieving, preventing or treating symptoms of the inflammatory disease and/or autoimmune disease. In other words, “treating” preferably refers to decreasing the duration (number of days/weeks/years the subject will suffer from the inflammatory disease and/or autoimmune disease), the risks, the complications and/or the severity of the inflammatory disease and/or autoimmune disease; this also encompasses the relief of the symptoms caused by the inflammatory disease and/or autoimmune disease.

The method for preventing and/or treating an inflammatory disease and/or autoimmune disease may hence be prophylactic, symptomatic and/or curative. In context of this disclosure, prophylactic, symptomatic and/or curative treatments may represent separate aspects of this disclosure.

Due to the sialylated saccharide according to this disclosure having little to no adverse effects in animals and humans, this form of therapy could be used as a preventive, as a first line therapy option, or as an adjunct to existing therapies that would be well tolerated by patients of either sex.

Method for Preventing and/or Treating

In an embodiment of the first aspect of this disclosure, a sialylated saccharide is for use in a method for preventing and/or treating an inflammatory disease and/or autoimmune disease in a subject.

In a preferred embodiment, the method comprises administering an effective amount of the sialylated saccharide (it is referred to in the Section “Saccharide”) to the subject. As understood by the skilled person, an “effective amount” is the amount of the sialylated saccharide that is required to confer a therapeutic effect on the subject as described in this disclosure. Effective amounts vary, as recognized by those skilled in the art, depending on the subject, route of administration, excipient usage among other known factors. Effective amounts may be determined empirically using known testing protocols or by extrapolation from in vivo or in vitro test data. It is further understood that for any particular subject/individual, specific dosage regimens should be adjusted over time according to the subject/individual need and the professional judgment of the person administering or supervising the administration of the products.

In a more preferred embodiment, the sialylated saccharide is administered at a daily dose of at least 1.0 mg, preferably at least 10.0 mg, more preferably at least 50.0 mg, even more preferably at least 100.0 mg, even more preferably at least 250.0 mg, even more preferably at least 500.0 mg, even more preferably at least 1.0 g, most preferably at least 1.5 g.

In the context of this disclosure, an amount of a sialylated saccharide according to this disclosure expressed in a number of grams or milligrams per daily dose as used herein means that the amount of the saccharide is such that when administering the daily dosage to a subject, the subject will be administered with the number of grams or milligrams of the saccharide. In other words, if the daily dosage is, for example, 50 mg, then the subject receives in total 50 mg per day. This may be in one or more portions. So, if the daily dosage is 50 mg divided over 2 portions, then a single serving consists of 25 mg, a daily serving consists of 2 of such single servings. Preferably, the daily dosage is administered in a single serving.

In an additional and/or alternative more preferred embodiment, the sialylated saccharide is administered at a daily dose of 0.001-15.0 g, preferably 0.001-12.5 g, more preferably 0.001-10.0 g, even more preferably 0.001-7.5 g, even more preferably 0.001-5.0 g, most preferably 0.001-2.5 g.

More preferably, the sialylated saccharide is administered at a daily dose of 0.010-15.0 g, preferably 0.010-12.5 g, more preferably 0.010-10.0 g, even more preferably 0.010-7.5 g, even more preferably 0.010-5.0 g, most preferably 0.010-2.5 g.

Even more preferably, the sialylated saccharide is administered at a daily dose of 0.050-15.0 g, preferably 0.050-12.5 g, more preferably 0.050-10.0 g, even more preferably 0.050-7.5 g, even more preferably 0.050-5.0 g, most preferably 0.050-2.5 g.

Even more preferably, the sialylated saccharide is administered at a daily dose of 0.100-15.0 g, preferably 0.100-12.5 g, more preferably 0.100-10.0 g, even more preferably 0.100-7.5 g, even more preferably 0.100-5.0 g, most preferably 0.100-2.5 g.

Even more preferably, the sialylated saccharide is administered at a daily dose of 0.250-15.0 g, preferably 0.250-12.5 g, more preferably 0.250-10.0 g, even more preferably 0.250-7.5 g, even more preferably 0.250-5.0 g, most preferably 0.250-2.5 g.

Even more preferably, the sialylated saccharide is administered at a daily dose of 0.500-15.0 g, preferably 0.500-12.5 g, more preferably 0.500-10.0 g, even more preferably 0.500-7.5 g, even more preferably 0.500-5.0 g, most preferably 0.500-2.5 g.

Even more preferably, the sialylated saccharide is administered at a daily dose of 1.0-15.0 g, preferably 1.0-12.5 g, more preferably 1.0-10.0 g, even more preferably 1.0-7.5 g, even more preferably 1.0-5.0 g, most preferably 1.0-2.5 g.

Most preferably, the sialylated saccharide is administered at a daily dose of 1.5-15.0 g, preferably 1.5-12.5 g, more preferably 1.5-10.0 g, even more preferably 1.5-7.5 g, even more preferably 1.5-5.0 g, most preferably 1.5-2.5 g.

In an additional and/or alternative more preferred embodiment, the sialylated saccharide is administered at a daily dose of 0.001-500.0 mg, preferably 0.001-250 mg, more preferably 0.001-200.0 mg, even more preferably 0.001-150.0 mg, even more preferably 0.001-125.0 mg, most preferably 0.001-100.0 mg, per kg bodyweight of the subject.

More preferably, the sialylated saccharide is administered at a daily dose of 0.01-500.0 mg, preferably 0.01-250 mg, more preferably 0.01-200.0 mg, even more preferably 0.01-150.0 mg, even more preferably 0.01-125.0 mg, most preferably 0.01-100.0 mg, per kg bodyweight of the subject.

Even more preferably, the sialylated saccharide is administered at a daily dose of 0.1-500.0 mg, preferably 0.1-250 mg, more preferably 0.1-200.0 mg, even more preferably 0.1-150.0 mg, even more preferably 0.1-125.0 mg, most preferably 0.1-100.0 mg, per kg bodyweight of the subject.

Even more preferably, the sialylated saccharide is administered at a daily dose of 1.0-500.0 mg, preferably 1.0-250 mg, more preferably 1.0-200.0 mg, even more preferably 1.0-150.0 mg, even more preferably 1.0-125.0 mg, most preferably 1.0-100.0 mg, per kg bodyweight of the subject.

Even more preferably, the sialylated saccharide is administered at a daily dose of 10.0-500.0 mg, preferably 10.0-250 mg, more preferably 10.0-200.0 mg, even more preferably 10.0-150.0 mg, even more preferably 10.0-125.0 mg, most preferably 10.0-100.0 mg, per kg bodyweight of the subject.

Even more preferably, the sialylated saccharide is administered at a daily dose of 25.0-500.0 mg, preferably 25.0-250 mg, more preferably 25.0-200.0 mg, even more preferably 25.0-150.0 mg, even more preferably 25.0-125.0 mg, most preferably 25.0-100.0 mg, per kg bodyweight of the subject.

Most preferably, the sialylated saccharide is administered at a daily dose of 50.0-500.0 mg, preferably 50.0-250 mg, more preferably 50.0-200.0 mg, even more preferably 50.0-150.0 mg, even more preferably 50.0-125.0 mg, most preferably 50.0-100.0 mg, per kg bodyweight of the subject.

In an additional and/or alternative more preferred embodiment, the sialylated saccharide is administered to the subject for at least 2, preferably at least 3, more preferably at least 4, even more preferably at least 6, even more preferably at least 8, even more preferably at least 10, most preferably at least 12, consecutive weeks. Preferably, the sialylated saccharide is administered to the subject for 2-32, preferably 2-32, more preferably 2-32, even more preferably 2-28, even more preferably 2-24, even more preferably 2-20, even more preferably 2-16, most preferably 2-12, consecutive weeks. More preferably, the sialylated saccharide is administered to the subject for 3-32, preferably 3-32, more preferably 3-32, even more preferably 3-28, even more preferably 3-24, even more preferably 3-20, even more preferably 3-16, most preferably 3-12, consecutive weeks. Even more preferably, the sialylated saccharide is administered to the subject for 4-32, preferably 4-32, more preferably 4-32, even more preferably 4-28, even more preferably 4-24, even more preferably 4-20, even more preferably 4-16, most preferably 4-12, consecutive weeks.

Preferably, the sialylated saccharide according to this disclosure is administered at least once a week (i.e., at least one daily dosage is administered in a week), more preferably at least once every 3 days, even more preferably at least once every 2 days, most preferably at least once daily (i.e., a daily dosage is administered every day).

The duration of treatment may be determined empirically using known testing protocols or by extrapolation from in vivo or in vitro test data. It is further understood that for any particular subject/individual, specific dosage regimens should be adjusted over time according to the subject/individual need and the professional judgment of the person administering or supervising the administration of the products.

It is also in the context of this disclosure that a treatment with a sialylated saccharide according to this disclosure consists of two or more treatment periods, wherein two consecutive treatment periods are interrupted for one or more weeks, preferably for at least 2 weeks, more preferably for at least 4 weeks, even more preferably for at least 8 weeks, most preferably for at least 12 weeks. Preferably, the treatment period consists of at least 2, more preferably at least 3, even more preferably at least 4, even more preferably at least 6, even more preferably at least 8, even more preferably at least 10, most preferably at least 12, consecutive weeks. More preferably, the treatment period consists of 2-32, preferably 2-32, more preferably 2-32, even more preferably 2-28, even more preferably 2-24, even more preferably 2-20, even more preferably 2-16, most preferably 2-12, consecutive weeks. Even more preferably, the treatment period consists of 3-32, preferably 3-32, more preferably 3-32, even more preferably 3-28, even more preferably 3-24, even more preferably 3-20, even more preferably 3-16, most preferably 3-12, consecutive weeks. Even more preferably, the treatment period consists of 4-32, preferably 4-32, more preferably 4-32, even more preferably 4-28, even more preferably 4-24, even more preferably 4-20, even more preferably 4-16, most preferably 4-12, consecutive weeks.

It has been found that a sialylated saccharide according to this disclosure (it is referred to in the Section “Saccharide”) is sufficient to obtain an effective therapeutic for preventing and/or treating an inflammatory disease or an autoimmune disease as described herein.

In this context, it is a preferred embodiment that one or more of the sialylated saccharides according to this disclosure (it is referred to in the Section “Saccharide”) is/are administered to the subject. It is a more preferred embodiment that two or more (most preferably only two) sialylated saccharides according to this disclosure (it is referred to in the Section “Saccharide”), i.e., a first sialylated saccharide according to this disclosure and a second sialylated saccharide according to this disclosure, are administered to the subject. Administering of additional saccharide(s) is not required to obtain the therapeutic effect but is not excluded from this disclosure.

When a first sialylated saccharide and a second sialylated saccharide are administered to the subject according to this disclosure, the first sialylated saccharide and the second sialylated saccharide are preferably administered on the same day, more preferably administered simultaneously. Simultaneous administration can be achieved by providing the first sialylated saccharide and the second sialylated saccharide in the same composition (the composition is preferably as described in the Section “Composition”).

In this context, it is a preferred embodiment that the amount of the first sialylated saccharide administered to the subject is 0.4 to 50.0, preferably 0.4 to 45.0, more preferably 0.4 to 40.0, even more preferably 0.4 to 35.0, most preferably 0.4 to 30.0, times the amount of the second sialylated saccharide administered to the subject. In a more preferred embodiment, the amount of the first sialylated saccharide administered to the subject is higher than the amount of the second sialylated saccharide, preferably the amount of the first sialylated saccharide is 1.1 to 50.0, preferably 1.1 to 45.0, more preferably 1.1 to 40.0, even more preferably 1.1 to 35.0, even more preferably 1.1 to 30.0, even more preferably 1.1 to 25.0, even more preferably 1.1 to 20.0, even more preferably 1.1 to 15.0, even more preferably 1.1 to 12.5, even more preferably 1.1 to 10.0, even more preferably 1.5 to 10.0, even more preferably 2.5 to 10, even more preferably 5.0 to 10.0, even more preferably 7.5 to 10.0, most preferably 8.0 to 10.0, times higher than the amount of the second sialylated saccharide. For the sake of clarity, the expression “x to y” as used throughout the disclosure and claims includes x, y and each value in between.

In this context, it is an additional and/or alternative preferred embodiment that the ratio between (i) the amount of the first sialylated saccharide administered to the subject and (ii) the amount of the second sialylated saccharide administered to the subject is similar to this ratio as found in the mother's milk of the same species. Throughout the disclosure and claims, unless specifically stated otherwise, a “similar” ratio preferably refers to a ratio that is identical or falls in the range of ratio +/−20% of the ratio, preferably ratio +/−10% of the ratio. For clarity, this means that when the subject is a dog, it is particularly preferred that the ratio between (i) the amount of the first sialylated saccharide being, for example, 3′SL administered to the subject and (ii) the amount of the second sialylated saccharide being, for example, 6′SL administered to the dog is similar to the 3′SL/6′SL ratio as generally found in mother's milk of a dog, i.e., 9:1+/−1.8 (in other words 7.2-10.9), preferably 9:1+/−0.9 (in other words 8.2-10.0).

When the subject is a human, then it is preferred that the ratio between (i) the amount of the first sialylated saccharide administered to the subject and (ii) the amount of the second sialylated saccharide administered to the subject is 0.4-4.0, preferably 0.4-3.5, more preferably 0.4-3.0, even more preferably 0.4-2.5, even more preferably 0.4-2.0, even more preferably 0.6-2.0, most preferably 1.0-2.0.

When the subject is a dog or horse, then it is preferred that the ratio between (i) the amount of the first sialylated saccharide administered to the subject and (ii) the amount of the second sialylated saccharide administered to the subject is 1.1 to 15.0, preferably 1.1 to 12.5, more preferably 1.1 to 10.0, even more preferably 1.5 to 10.0, even more preferably 2.5 to 10, even more preferably 5.0 to 10.0, even more preferably 7.5 to 10.0, most preferably 8.0 to 10.0.

When the subject is a bovine, then it is preferred that the ratio between (i) the amount of the first sialylated saccharide administered to the subject and (ii) the amount of the second sialylated saccharide administered to the subject is 1.5 to 6.0, preferably 1.5 to 5.0, more preferably 1.5 to 4.0, even more preferably 1.5 to 3.5, even more preferably 2.0 to 3.5, most preferably 2.5 to 3.5.

When the subject is a pig or a cat, then it is preferred that the ratio between (i) the amount of the first sialylated saccharide administered to the subject and (ii) the amount of the second sialylated saccharide administered to the subject is 7.5-40.0, preferably 7.5-35.0, more preferably 10.0-35.0, even more preferably 15.0-35.0, even more preferably 20.0-35.0, most preferably 25.0-35.0.

When the subject is a goat, then it is preferred that the ratio between (i) the amount of the first sialylated saccharide administered to the subject and (ii) the amount of the second sialylated saccharide administered to the subject is 0.25-5.0, preferably 0.25-3.0, more preferably 0.25-2.0, even more preferably 0.25-1.5, most preferably 0.5-1.5.

In certain embodiments, the method for preventing and/or treating an inflammatory disease and/or autoimmune disease in a subject according to this disclosure can be combined with the administration of one or more anti-inflammatory agents as known in the art including, but not limited to, nonsteroidal anti-inflammatory drugs, glucocorticoids, biologic response modifiers and opioids.

Examples of nonsteroidal anti-inflammatory drugs include Aminophenazone, Ampyrone, Azapropazone, Clofezone, Difenamizole, Famprofazone, Feprazone, Kebuzone, Metamizole, Mofebutazone, Morazone, Nifenazone, Oxyphenbutazone, Phenazone, Phenylbutazone, Propyphenazone, Sulfinpyrazone, Suxibuzone, Aspirin, Aloxiprin, Benorylate, Carbasalate, calcium Diflunisal, Dipyrocetyl, Ethenzamide, Guacetisal, Magnesium salicylate, Methyl salicylate, Salsalate, Salicin, Salicylamide, Salicylic acid (salicylate), Sodium salicylate, Aceclofenac, Acemetacin, Alclofenac, Amfenac, Bendazac, Bromfenac, Bumadizone, Bufexamac, Diclofenac, Difenpiramide, Etodolac, Felbinac, Fenclozic acid, Fentiazac, Indomethacin, Indomethacin farnesil, Isoxepac, Ketorolac, Lonazolac, Oxametacin, Prodolic acid, Proglumetacin, Sulindac, Tiopinac, Tolmetin, Zomepirac, Ampiroxicam, Droxicam, Isoxicam, Lornoxicam, Meloxicam, Piroxicam, Tenoxicam, Alminoprofen, Benoxaprofen, Carprofen, Dexibuprofen, Dexketoprofen, Fenbufen, Fenoprofen, Flunoxaprofen, Flurbiprofen, Ibuprofen, Ibuproxam, Indoprofen, Ketoprofen, Loxoprofen, Miroprofen, Naproxen, Oxaprozin, Pirprofen, Suprofen, Tarenflurbil, Tepoxalin, Tiaprofenic acid, Vedaprofen, Naproxcinod, Azapropazone, Clonixin, Etofenamate, Flufenamic acid, Flunixin, Meclofenamic acid, Mefenamic acid, Morniflumate, Niflumic acid, Tolfenamic acid, Flutiazin, Apricoxib, Celecoxib, Cimicoxib, Deracoxib, Etoricoxib, Firocoxib, Lumiracoxib, Mavacoxib, Parecoxib, Robenacoxib, Rofecoxib, Valdecoxib, Aminopropionitrile, Benzydamine, Chondroitin sulfate, Diacerein, Fluproquazone, Glucosamine, Glycosaminoglycan, Hyperforin, Nabumetone, Nimesulide, Oxaceprol, Proquazone, Superoxide dismutase/Orgotein, and Tenidap.

Examples of glucocorticoids include betamethasone and prednisone.

Examples of biologic response modifiers include hydroxychloroquine, leflunomide, methotrexate, tofacitinib, abatacept, adalimumab, adalimumab-atto, anakinra, etanercept, etanercept-szzs, rituximab, infliximab-dyyb, golimumab, certolizumab pegol, tocilizumab, and sarilumab.

Examples of opioids include tramadol, oxycontin, oxycodone, fentanyl, morphine, codeine, dihydrocodeine and actiq.

### Inflammatory Disease/Autoimmune Disease

In an embodiment of the first aspect of this disclosure, a sialylated saccharide is for use in a method for preventing and/or treating an inflammatory disease and/or autoimmune disease in a subject.

In the context of this disclosure, the term “disease” refers to an abnormal condition of the body, or one or more of its parts, of a subject that impairs normal functioning and that is typically manifested by distinguishing signs and symptoms. The term “inflammatory disease” refers to a disease characterized by inflammation where the subject fails to return to his original healthy state. It is usually mediated by an inflammatory cytokine cascade, i.e., an in vivo release from cells of at least one pro-inflammatory cytokine in a subject wherein the cytokine release affects a physiological condition of the subject. Inflammatory disease is well-known to the skilled person as evidenced by, for example, the textbook entitled “Compendium of Inflammatory Diseases” (published by Springer Basel, 2016, Michael J. Parnham as editor, ISBN 978-3-7643-8530-9). The term “autoimmune disease” refers to a disease wherein the subject's own immune system attacks a healthy part of the body by mistake, in other words the subject's immune system does not distinguish between healthy tissue and potentially harmful antigens. Over 100 autoimmune diseases are currently known as enlisted in the Autoimmune Disease List from the Autoimmune Association (autoimmune.org/disease-information). Throughout the disclosure and claims, the term “inflammatory disease” is preferably replaced with “chronic inflammatory disease,” unless specifically stated otherwise.

Preferably, the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, antiphospholipid antibody syndrome, myositis, scleroderma, vasculitis, Sjogren's syndrome, asthma, tuberculosis, chronic periodontitis, chronic sinusitis, chronic active hepatitis, Alzheimer's, Parkinson's disease, nephritis, fibromyalgia, atherosclerosis, eczema, gastritis, sinusitis, seborrheic dermatitis, type-2 diabetes and Wegener's granulomatosis.

More preferably, the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, atherosclerosis and type-2 diabetes.

Even more preferably, the inflammatory disease is selected from the list consisting of osteoarthritis, rheumatoid arthritis, psoriatic arthritis, juvenile idiopathic arthritis, ankylosing spondylitis, systemic lupus erythematosus, gout and enteropathic arthritis.

Even more preferably, the inflammatory disease is selected from the list consisting of osteoarthritis, rheumatoid arthritis, psoriatic arthritis, juvenile idiopathic arthritis, ankylosing spondylitis, systemic lupus erythematosus and gout.

Even more preferably, the inflammatory disease is selected from the list consisting of osteoarthritis, rheumatoid arthritis, psoriatic arthritis, juvenile idiopathic arthritis, ankylosing spondylitis and systemic lupus erythematosus.

Even more preferably, the inflammatory disease is selected from the list consisting of osteoarthritis, rheumatoid arthritis and psoriatic arthritis.

Most preferably, the inflammatory disease is osteoarthritis or rheumatoid arthritis.

Preferably, the autoimmune disease is selected form the list consisting of autoimmune myocarditis, Dressier's syndrome, postpericardiotomy syndrome, subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, Systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune Oophoritis, endometriosis, Sjogren syndrome, autoimmune enteropathy, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, chronic Lyme disease, mixed connective tissue disease, palindromic rheumatism, Parry Romberg syndrome, Parsonage-Turner syndrome, psoriatic arthritis, reactive arthritis, relapsing polychondritis, retroperitoneal fibrosis, rhematic fever, rheumatoid arthritis, sarcoidosis, Schnitzler syndrome, systemic lupus erythematosus, undifferentiated connective tissue disease, dermatomyositis, fibromyalgia, inclusion body myositis, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, autoimmune inner ear disease, Meniere's disease, Behget's disease, eosinophilic granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatica, urticarial vasculitis, vasculitis, and primary immune deficiency.

More preferably, the autoimmune disease is selected from the list consisting of rheumatoid arthritis, psoriatic arthritis, juvenile arthritis, ankylosing spondylitis, systemic lupus erythematosus, reactive arthritis, drug-induced lupus, enthesitis-related arthritis, lupus vasculitis, rheumatoid vasculitis and diabetes mellitus type 1.

Even more preferably, the autoimmune disease is selected from the list consisting of rheumatoid arthritis, psoriatic arthritis, juvenile arthritis, ankylosing spondylitis, systemic lupus erythematosus, reactive arthritis, drug-induced lupus, enthesitis-related arthritis, and diabetes mellitus type 1.

Even more preferably, the autoimmune disease is selected from the list consisting of rheumatoid arthritis, psoriatic arthritis, juvenile arthritis, ankylosing spondylitis, systemic lupus erythematosus, reactive arthritis, drug-induced lupus and enthesitis-related arthritis.

Even more preferably, the autoimmune disease is selected from the list consisting of rheumatoid arthritis, psoriatic arthritis, juvenile arthritis, ankylosing spondylitis, systemic lupus erythematosus, reactive arthritis and enthesitis-related arthritis.

Most preferably, the autoimmune disease is rheumatoid arthritis, psoriatic arthritis and juvenile arthritis.

In a preferred embodiment according to this disclosure, a sialylated saccharide is for use in a method for preventing and/or treating an inflammatory disease and/or autoimmune disease in a subject, wherein the inflammatory disease and/or autoimmune disease is related to an organ. In an additional and/or alternative preferred embodiment, a sialylated saccharide is for use in a method for preventing and/or treating an inflammatory disease and/or autoimmune disease in a subject, wherein the disease affects any one or more selected from the list consisting of joint, bone, lung, liver, kidney, heart, intestine and colon, preferably any one or more selected from the list consisting of joint, bone, lung, liver, kidney and heart, even more preferably any one or more selected from the list consisting of joint, bone, lung, liver and kidney, even more preferably any one or more selected from the list consisting of joint, bone and lung, most preferably joint.

Preferably, the inflammatory disease affecting the joint is selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis.

Preferably, the autoimmune disease affecting the joint is selected from the list consisting of rheumatoid arthritis, psoriatic arthritis, juvenile idiopathic arthritis, ankylosing spondylitis, systemic lupus erythematosus, juvenile arthritis, reactive arthritis and enthesitis-related arthritis.

In a more preferred embodiment according to this disclosure, a sialylated saccharide is for use in a method for preventing and/or treating a disease that causes joint inflammation. The “sialylated saccharide” is as described in the Section “Saccharide,” the “method for preventing and/or treating” is as described in the Section “Method for preventing and/or treating.” The subject is further elaborated in the Section “Subject.”

Preferably, the disease that causes joint inflammation is selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis.

In an additional and/or alternative more preferred embodiment, the sialylated saccharide is for use in a method for preventing and/or treating arthritis.

Preferably, the arthritis is selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis.

Preferably, the arthritis is selected from the list consisting of osteoarthritis, rheumatoid arthritis, psoriatic arthritis, systemic lupus erythematosus, juvenile arthritis and gout.

More preferably, the arthritis is selected from the list consisting of osteoarthritis, rheumatoid arthritis, psoriatic arthritis, systemic lupus erythematosus and juvenile arthritis.

Even more preferably, the arthritis is selected from the list consisting of osteoarthritis, rheumatoid arthritis, systemic lupus erythematosus and juvenile arthritis.

In the context of this disclosure, the term “osteoarthritis (OA)” refers to the most prevalent type of arthritis, particularly in adults 65 years and older. The disease is characterized by chronic degenerative arthropathy that frequently leads to chronic pain and disability. The reported incidence and prevalence rates of OA in specific joints vary widely, due to differences in the case definition of OA. For example, OA may be defined by radiographic criteria alone (radiographic OA), by typical symptoms (symptomatic OA), or by both. Using radiographic criteria, the distal and proximal interphalangeal joints of the hand have been identified as the joints most commonly affected, but they are the least likely to be symptomatic. In contrast, the knee and hip, which constitute the second and third most common locations of radiographic OA, respectively, are nearly always symptomatic. The first metatarsal phalangeal and carpometacarpal joints are also frequent sites of radiographic OA, while the shoulder, elbow, wrist and metacarpophalangeal joints rarely develop idiopathic OA. Age is the most consistently identified risk factor for OA and prevalence rates rise steeply after age 50 in men and age 40 in women. OA is diagnosed by a triad of typical symptoms, physical findings and radiographic changes. The American College of Rheumatology has set forth classification criteria to aid in the identification of patients with symptomatic OA that include, but do not rely solely on, radiographic findings. Patients with early disease experience localized joint pain that worsens with activity and is relieved by rest, while those with severe disease may have pain at rest. Weight bearing joints may “lock” or “give way” due to internal derangement that is a consequence of advanced disease. Stiffness in the morning or following inactivity (“gel phenomenon”) rarely exceeds 30 minutes. Physical findings in osteoarthritic joints include bony enlargement, crepitus, cool effusions, and decreased range of motion. Tenderness on palpation at the joint line and pain on passive motion are also common, although not unique to OA. Radiographic findings in OA (slide) include osteophyte formation, joint space narrowing, subchondral sclerosis and cysts. The presence of an osteophyte is the most specific radiographic marker for although it is indicative of relatively advanced disease. Radiographs are considered the “gold standard” test for the diagnosis of OA, but radiographic changes are evident only relatively late in the disease. Routine laboratory studies, such as sedimentation rates and c-reactive protein, are not useful as markers for OA, although a recent study suggests that elevation of CRP predicts more rapidly progressive disease. Several epitopes of cartilage components, however, have been described that offer some promise as markers of OA. For example, chondroitin sulfate epitope 846, normally expressed only in fetal and neonatal cartilage, has been observed in OA, but not normal adult, cartilage and synovial fluid. Along a similar vein, an epitope unique to type II collagen has been described in OA cartilage and can be unmasked in vitro by exposing normal cartilage to MMPs. This epitope can be measured in blood and urine and may prove useful in diagnosing or monitoring OA progression. Elevated serum hyaluronan levels have also been shown by some to correlate with radiographic OA. The finding of elevated cartilage oligomeric protein (COMP) levels in synovial fluid after traumatic joint injury may portend development of OA in the injured joint. Other potential markers of OA are listed but are either not easily accessible or lack the sensitivity and specificity required to consider them as potential OA markers. Clinical symptoms of inflammation, the presence of histological inflammation in OA synovial tissue and early cartilage lesions at the border of the inflamed synovium are strong indicators that synovitis is a pivotal factor in the pathogenesis of OA. The traditional view of OA as a cartilage only disease is obsolete. OA is now considered to be a whole joint disease that includes the synovial tissue. Bone, cartilage and synovium communicate by way of cell-cell interactions, through the release of soluble mediators and via mechanical signals. Synovial inflammation, despite not being a prerequisite for the development of OA, is clearly involved in cartilage breakdown and thus in the progression of the disease. Animal models for osteoarthritis include mouse, rat, guinea pig, rabbit, dog, sheep, goat, mini pig and horse. Joint mechanics, cartilage thickness and histology, and OA disease progression rates are variable among the species and joints being studied. As large mammals, the larger animal models (dog, sheep, goat, horse and pig) generally have cartilage morphology and responses to injury that are more similar to humans and are hence used to study the disease process and treatment (Teeple et al, 2013, AAPS J. 15(2): p. 438-446; Kuyinu et al, 2016, J. Orthop. Surg. Res. 11(19): p. 1-27). Further, the dog and horse animal models as these are so-called spontaneous models and hence closely simulate the natural progression of human primary osteoarthritis (Kuyinu et al, supra). An established and reliable non-invasive method to screen and evaluate osteoarthritis in dogs is force plate analysis (Moreau et al., 2003, Veterinary Record 152(11): p. 323-329; Mueller et al., 2007, Veterinary Record 160(22): p. 762-765; Brown et al., 2013, J. Veterinary Internal Medicine 27(1): p. 22-30). Force plate analysis has been successfully used in dogs to evaluate lameness and/or the effects of treatment in a non-invasive, objective fashion (Kalis et al., 2012, Vet. Surg. 41(1): p. 148-155; Smolders et al., 2012, Vet. Surg. 41(6): p. 720-732; Van der Peijl et al., 2012, Vet. Comp. Orthop. Traumatol. 25(2): p. 126-134). After the publication of a study by McLaughlin et al. (1991, Vet. Surg. 20(5): p. 291-297), force plate analysis became an objective standard in the evaluation of canine locomotion.

In the context of this disclosure, the term “rheumatoid arthritis (RA)” refers to a systemic disease that affects the entire body and is one of the most common forms of arthritis. It is characterized by inflammation of the joint, which causes pain, stiffness, warmth, redness and swelling. This inflammation is a consequence of inflammatory cells invading the joints and these inflammatory cells release enzymes that may digest bone and cartilage. As a result, this inflammation can lead to severe bone and cartilage damage and to joint deterioration and severe pain amongst other physiologic effects. The involved joint can lose its shape and alignment, resulting in pain and loss of movement. There are several animal models for rheumatoid arthritis known in the art (Wooley, 2008, Current Rheumatology Reviews 4: p. 277-287). For example, in the collagen-induced arthritis (CIA) model, mice develop an inflammatory arthritis that resembles human rheumatoid arthritis. Since CIA shares similar immunological and pathological features with RA, this makes it a suitable model for screening potential human anti-inflammatory compounds. Efficacy in this model is measured by decrease in joint swelling. Efficacy in RA in the clinic is measured by the ability to reduce symptoms in patients, which is measured as a combination of joint swelling, erythrocyte sedimentation rate, C-reactive protein levels and levels of serum factors.

In the context of this disclosure, the term “psoriatic arthritis” refers to a type of inflammatory arthritis that occurs in a subset of patients with psoriasis. In these patients, the skin pathology/symptoms are accompanied by joint swelling, similar to that seen in rheumatoid arthritis. It features patchy, raised, red areas of skin inflammation with scaling. Psoriasis often affects the tips of the elbows and knees, the scalp, the navel, and around the genital areas or anus. The term “psoriatic arthritis” denotes a heterogenous group of arthritides ranging from peripheral monoarticular, oligoarticular and polyarticular disease, to axial skeletal involvement. Yet, despite this apparent clinical heterogeneity, these various presentations are unified in their occurrence in individuals with cutaneous manifestations of psoriasis, rheumatoid factor sero-negativity, similar human leukocyte antigen (HLA) associations and radiographic similarities. Approximately 2% of the Caucasian population in North America has psoriasis. Of these, 5-7% are affected by an inflammatory arthritis in some form. Psoriatic skin disease pre-dates the onset of arthritis in 70% of cases, presents coincident with arthritis in 15% of cases, and follows the onset of arthritis in 15% of cases. The cause of psoriatic arthritis is unknown. The inflammatory and autoimmune character of the disease is supported not only by the clinical presentation, but also by the role that T-cells and various cytokines have been demonstrated to play in both the initiation and perpetuation of disease activity.

In the context of this disclosure, the terms “systemic lupus erythematosus,” “SLE” and “lupus” are interchangeably used herein. Systemic lupus erythematosus is an autoimmune disease in which the immune system attacks its own tissues, causing widespread inflammation and tissue damage in the affected organs. It can affect joint, skin, brain, lung, kidney and blood vessel. It is characterized by antibodies to nuclear and cytoplasmic antigens, multisystem inflammation, protean clinical manifestations, and a relapsing and remitting course. More than 90% of the cases occur in women, often at childbearing age. Most frequent symptoms include pain or swelling in the joints, fatigue, skin rashes and fever. Other symptoms include arthritis, sun sensitivity, oral ulcers, lung problems, heart problems, kidney problems, seizures, blood cell and immunological abnormalities. Using symptom assessments, physical examination, X-rays and lab tests SLE is diagnosed.

In the context of this disclosure, the term “juvenile arthritis,” also known as “childhood arthritis” refers to inflammatory and rheumatic diseases that develop in children under the age of 16, i.e., arthritis in children under the age of 16. It is an umbrella term that covers several diseases including juvenile idiopathic arthritis (most common form of juvenile arthritis and is a preferred embodiment of juvenile arthritis in this disclosure).

In the context of this disclosure, the term “gout” refers to a type of arthritis that causes sudden, severe and recurrent attacks of joint pain and is caused by the deposition of monosodium urate monohydrate crystals. Most often the big toe is affected, but it can also strike other joints, such as other toes, ankle or knee. Gout is the most common type of inflammatory arthritis and mostly develops in people who have high levels of uric acid from the breakdown of purines, which are found in human cells and many foods, including red meat, organ meat and some seafoods. The diagnosis of gout includes joint fluid analysis (microscopic analysis for uric acid crystals), blood test to check uric acid level and imaging tests (e.g., X-ray, ultrasound, MRI and dual-energy computerized tomography) to visualize uric acid crystals in the joints.

In an even more preferred embodiment, the inflammatory disease and the autoimmune disease is not a disease associated with the digestive tract.

In an additional and/or alternative even more preferred embodiment, the inflammatory disease and the autoimmune disease is not degenerative arthritis.

### Mode of Action

It has been found that a sialylated saccharide according to this disclosure promotes the polarization of peripheral blood mononuclear CD14+ cells (preferably monocytes) toward a less inflammatory status. As understood by the skilled person and in the context of this disclosure, the expression “toward a less inflammatory status” refers to a decrease of the ratio between inflammatory monocytes and patrolling monocytes in the blood.

More specifically, it has been found that a sialylated saccharide according to this disclosure is an efficacious agent to prevent and/or treat an inflammatory disease and/or an autoimmune disease in a subject by downregulating the tumor necrosis factor alpha (TNF-α) signaling via NF-κB (nuclear factor kappa B) pathway and/or downregulating the IL-6/JAK/STAT3 signaling pathway in peripheral blood mononuclear CD14+ cells. The CD14+ cells are preferably monocytes.

This technical effect identifies a new clinical situation, namely one in which it could be preferable to target the systemic immunity of a subject rather than the tissue inflammation (e.g., joint inflammation) or the tissue damage/repair (e.g., cartilage destruction or repair). Monocytes are an interesting target as they play an important role in the pathogenesis and progression of chronic inflammatory diseases such as osteoarthritis (Sejourne et al, 2017, Joint Bone Spine 84(2): p. 175-181; Loukov et al, 2018, Osteoarthritis Cartilage 26(2): p. 255-263; Shi et al, 2019, Biomed. Res. Int.: 4291689; Haubruck et al, 2021, Front. Immunol. 12: 763702).

This technical effect can be assessed in a subject by the skilled person by using routinely available techniques in the art. Peripheral blood mononuclear cells can be isolated from a blood sample using standard techniques such as density gradient centrifugation (e.g., as illustrated in the Examples below). Subsequently, CD14+ cells can be positively selected from the isolated peripheral blood mononuclear cells by well-known isolation methods such as using antibodies that are directed to the CD14 epitope.

The tumor necrosis factor alpha (TNF-α) signaling via NF-κB (nuclear factor kappa B) pathway and IL-6/JAK/STAT3 signaling pathway in isolated peripheral blood mononuclear CD14+ cells can be assessed using whole transcriptome followed by pathway enrichment or quantification of pathway marker genes. In a first preferred method, differential expression analysis of the whole transcriptome is followed by Gene Set Enrichment Analysis using GSEA function in clusterProfiler (bioconductor.org/packages/release/bioc/html/clusterProfiler.html) of the ranked genes (based on −log 10(p-value)*sign(fold change)) and the Hallmark genesets via the msigdbr package (github.com/igordot/msigdbr). In a second preferred method, one or more genes with abs(log 2FC)>=1 is differential expressed from HALLMARK_TNFA_SIGNALING_VIA_NFKB geneset including ABCA1, AREG, ATF3, ATP2B1, B4GALT1, B4GALT5, BCL2A1, BCL3, BCL6, BHLHE40, BIRC2, BIRC3, BMP2, BTG1, BTG2, BTG3, CCL2, CCL20, CCL4, CCL5, CCND1, CCNL1, CCRL2, CD44, CD69, CD80, CD83, CDKN1A, CEBPB, CEBPD, CFLAR, CLCF1, CSF1, CSF2, CXCL1, CXCL10, CXCL11, CXCL2, CXCL3, CXCL6, ACKR3, CCN1, DDX58, DENND5A, DNAJB4, DRAM1, DUSP1, DUSP2, DUSP4, DUSP5, EDN1, EFNA1, EGR1, EGR2, EGR3, EHD1, EIF1, ETS2, F2RL1, F3, FJX1, FOS, FOSB, FOSL1, FOSL2, FUT4, GOS2, GADD45A, GADD45B, GCH1, GEM, GFPT2, GPR183, HBEGF, HES1, ICAM1, ICOSLG, ID2, IER2, IER3, IER5, IFIH1, IFIT2, IFNGR2, IL12B, IL15RA, IL18, ILlA, IL1B, IL23A, IL6, IL6ST, IL7R, INHBA, IRF1, IRS2, JAG1, JUN, JUNB, KDM6B, KLF10, KLF2, KLF4, KLF6, KLF9, KYNU, LAMB3, LDLR, LIF, LITAF, MAFF, MAP2K3, MAP3K8, MARCKS, MCL1, MSC, MXD1, MYC, NAMPT, NFAT5, NFE2L2, NFIL3, NFKB1, NFKB2, NFKBIA, NFKBIE, NINJ1, NR4A1, NR4A2, NR4A3, OLR1, PANX1, PDE4B, PDLIM5, PER1, PFKFB3, PHLDA1, PHLDA2, PLAU, PLAUR, PLEK, PLK2, PMEPA1, PNRC1, PLPP3, PPP1R15A, PTGER4, PTGS2, PTPRE, PTX3, RCAN1, REL, RELA, RELB, RHOB, RIPK2, RNF19B, SAT1, SDC4, SERPINB2, SERPINB8, SERPINEl, SGK1, SIK1, SLC16A6, SLC2A3, SLC2A6, SMAD3, SNN, SOCS3, SOD2, SPHK1, SPSB1, SQSTM1, STAT5A, TANK, TAP1, TGIF1, TIPARP, TLR2, TNC, TNF, TNFAIP2, TNFAIP3, TNFAIP6, TNFAIP8, TNFRSF9, TNFSF9, TNIP1, TNIP2, TRAF1, TRIB1, TRIP10, TSC22D1, TUBB2A, VEGFA, YRDC, ZBTB10, ZC3H12A, ZFP36 or HALLMARK_IL6_JAK_STAT3_SIGNALING geneset including A2M, ACVR1B, ACVRL1, BAK1, CBL, CCL7, CCR1, CD14, CD36, CD38, CD44, CD9, CNTFR, CRLF2, CSF1, CSF2, CSF2RA, CSF2RB, CSF3R, CXCL1, CXCL10, CXCL11, CXCL13, CXCL3, CXCL9, DNTT, EBI3, FAS, GRB2, HAX1, HMOX1, IFNAR1, IFNGR1, IFNGR2, IL10RB, IL12RB1, IL13RA1, IL15RA, IL17RA, IL17RB, IL18R1, IL1B, IL1R1, IL1R2, IL2RA, IL2RG, IL3RA, IL4R, IL6, IL6ST, IL7, IL9R, INHBE, IRF1, IRF9, ITGA4, ITGB3, JUN, LEPR, LTB, LTBR, MAP3K8, MYD88, OSMR, PDGFC, PF4, PIK3R5, PIM1, PLA2G2A, PTPN1, PTPN11, PTPN2, REGlA, SOCS1, SOCS3, STAM2, STAT1, STAT2, STAT3, TGFB1, TLR2, TNF, TNFRSF12A, TNFRSFlA, TNFRSF1B, TNFRSF21, TYK2. The assessment of the tumor necrosis factor alpha (TNF-α) signaling via NF-κB (nuclear factor kappa B) pathway and IL-6/JAK/STAT3 signaling pathway in isolated peripheral blood mononuclear CD14+ cells is illustrated in the Examples below.

In a preferred embodiment, the tumor necrosis factor alpha (TNF-α) signaling via NF-κB (nuclear factor kappa B) pathway decreases (i.e., is downregulated) with an absolute normalized enrichment score (NES) of at least 1.0, preferably at least 1.1, more preferably at least 1.2, even more preferably at least 1.3, most preferably at least 1.4, preferably over a period of time. The period of time is preferably 2 weeks, more preferably 1 month, even more preferably 2 months, even more preferably 3 months, even more preferably 6 months, even more preferably 9 months, even more preferably 12 months. Throughout the disclosure and claims, the period of time is preferably the duration of the method according to this disclosure (it is referred to in the Section “Method for preventing and/or treating”). The decrease can be relative to the tumor necrosis factor alpha (TNF-α) signaling via NF-κB (nuclear factor kappa B) pathway of the subject prior to administration of the sialylated saccharide or can be relative to the tumor necrosis factor alpha (TNF-α) signaling via NF-κB (nuclear factor kappa B) pathway of a control healthy subject that did not receive the sialylated saccharide. Preferably, the increase is relative to the tumor necrosis factor alpha (TNF-α) signaling via NF-κB (nuclear factor kappa B) pathway of the subject prior to administration of the sialylated saccharide.

In an additional and/or alternative preferred embodiment, the IL-6/JAK/STAT3 signaling pathway decreases (i.e., is downregulated) with an absolute NES of at least 1.0 preferably at least 1.1, more preferably at least 1.2, even more preferably at least 1.3, most preferably at least 1.4, preferably over a period of time. The period of time is preferably 2 weeks, more preferably 1 month, even more preferably 2 months, even more preferably 3 months, even more preferably 6 months, even more preferably 9 months, even more preferably 12 months. Throughout the disclosure and claims, the period of time is preferably the duration of the method according to this disclosure (it is referred to in the Section “Method for preventing and/or treating”). The decrease can be relative to the IL-6/JAK/STAT3 signaling pathway of the subject prior to administration of the sialylated saccharide or can be relative to the IL-6/JAK/STAT3 signaling pathway of a control healthy subject that did not receive the sialylated saccharide. Preferably, the increase is relative to the IL-6/JAK/STAT3 signaling pathway of the subject prior to administration of the sialylated saccharide.

### Subject

In a preferred embodiment of the first aspect of this disclosure, the sialylated saccharide is for use in a method for preventing and/or treating an inflammatory disease and/or autoimmune disease in a subject, wherein the subject is a human or an animal.

In a more preferred embodiment, the sialylated saccharide is for use in a method for preventing and/or treating an inflammatory disease and/or autoimmune disease in a subject, wherein the subject is a human or a mammal.

Preferably, the mammal is a farmed animal or a domesticated animal, more preferably a domesticated animal.

As understood by the skilled person, a “farmed animal” refers to an animal that is reared in an agricultural setting in order to produce various commodities such as food (meat, organs, eggs, dairy products) and/or hair or wool. A farmed animal is preferably selected from the list consisting of bovine, pig, sheep, goat, horse, camelid (preferably llama or alpaca) and rabbit.

As understood by the skilled person, a “domesticated animal” refers to an animal that has been selectively bred and adapted over generations to live alongside humans. A domesticated animal in the context of this disclosure is preferably a companion animal or a horse; more preferably the domesticated animal is selected from the list consisting of a dog, a cat and a horse.

In an even more preferred embodiment, the sialylated saccharide according to this disclosure is for use in a method for preventing and/or treating an inflammatory disease and/or autoimmune disease in a human.

### Saccharide

In an embodiment of the first aspect of this disclosure, the sialylated saccharide is for use in a method for preventing and/or treating an inflammatory disease and/or autoimmune disease in a subject.

A “sialylated saccharide” refers to a saccharide that comprises a sialic acid, preferably a N-acetylneuraminic acid (i.e., Neu5Ac). Preferably, the sialylated saccharide according to this disclosure contains only one sialic acid (preferably only one Neu5Ac), i.e., the saccharide consists of one or more monosaccharides and only one of the monosaccharides is a sialic acid (preferably a Neu5Ac). As understood by the skilled person, the term “saccharide” refers to a molecule comprising at least one monosaccharide, in other words a saccharide is a molecule consisting of one or more monosaccharide residue(s). The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar.

In an additional and/or alternative embodiment, the sialylated saccharide comprises a sialic acid, preferably Neu5Ac, that is linked to a monosaccharide in an alpha-2,3-, alpha-2,6- or alpha-2,8-linkage, preferably an alpha-2,3- or an alpha-2,6-linkage, more preferably an alpha-2,3-linkage, and wherein the monosaccharide is preferably selected from galactose, N-acetylglucosamine and Neu5Ac, more preferably the monosaccharide is galactose or N-acetylglucosamine, even more preferably the monosaccharide is galactose. The skilled person will understand that the expression “sialic acid is linked to a monosaccharide” refers to the situation wherein the sialic acid is bound to the monosaccharide through a glycosidic bond and wherein the sialic acid and monosaccharide are part of the saccharide of this disclosure (which can comprise additional monosaccharide(s) than the sialic acid and the monosaccharide).

In a preferred embodiment, the saccharide is a disaccharide or an oligosaccharide. In a more preferred embodiment, the saccharide is an oligosaccharide. In the context of this disclosure, the term “oligosaccharide” preferably refers to a saccharide containing 2 up to and including 20 monosaccharides, i.e., the degree of polymerization (DP) is 2-20. An oligosaccharide can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. Each monosaccharide can be in the cyclic form (e.g., pyranose or furanose form). An oligosaccharide can contain both alpha- and beta-glycosidic bonds or can contain only beta-glycosidic bonds.

More preferably, the oligosaccharide consists of 3-9, preferably 3-8, more preferably 3-7, even more preferably 3-6, most preferably 3-5, monosaccharides. For the sake of clarity, the expression “x-y” refers to a range from and including x to and including y. For example, 3-5 monosaccharides means that 3, 4 or 5 monosaccharides are present.

In an additional and/or alternative preferred embodiment, the sialylated saccharide according to this disclosure is a milk saccharide and/or a Lewis-type antigen saccharide.

Preferably, the Lewis-type antigen saccharide is sialyl Lewis a or sialyl Lewis x.

In a more preferred embodiment, the sialylated saccharide, according to this disclosure, is a milk saccharide, i.e., a saccharide that is found in milk of an animal, preferably a mammal and/or a human. In the context of this disclosure, it is preferred that the sialylated saccharide according to this disclosure is a milk oligosaccharide, i.e., a sialylated oligosaccharide that is found in milk of an animal, preferably a mammal and/or human.

Preferably, the milk saccharide/milk oligosaccharide is a mammalian milk saccharide/mammalian milk oligosaccharide (MMO). More preferably, the milk saccharide/milk oligosaccharide is a human milk saccharide/human milk oligosaccharide (HMO).

As understood by the skilled person, mammalian milk oligosaccharides (MMOs) comprise oligosaccharides present in milk found in any phase during lactation including colostrum milk from humans (i.e., human milk oligosaccharides or HMOs) and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus) (Urashima T. et al., 2011, Milk Oligosaccharides, Nova Biomedical Books, New York ISBN 978-1-61122-831-1; Coppa et al, 2013, Ital. J. Pediatr. 2013, 39(2)). A replete amount of milk saccharide structures has been elucidated so far. The majority of milk oligosaccharides found in animals such as mammals and humans comprise lactose at the reducing end (Urashima et al, 2011). Other milk oligosaccharides comprise N-acetyllactosamine (Gal-β1,4-GlcNAc) or lacto-N-biose (Gal-β1,3-GlcNAc) at the reducing end (Urashima et al, 2011; Wrigglesworth et al, 2020, PLoS ONE 15(12); Urashima et al, 2013, Biosci. Biotechnol. Biochem 77(3): p. 455-466; Wei et al, 2018, Sci. Rep. 8:4688). Examples hereof are 3-FLN (Gal-β1,4-(Fuc-α1,3-)GlcNAc; also known as Lewis x antigen), 3′-SLN (Neu5Ac-α2,3-Gal-β1,4-GlcNAc), 6′-SLN (Neu5Ac-α2,6-Gal-β1,4-GlcNAc) (Urashima et al, 2011; Wrigglesworth et al, 2020; Wei et al, 2018). Further, milk saccharides comprise milk glycosaminoglycans (GAGs; Coppa et al, 2013; Rai et al, 2021, Int. J. Biol. Macromolecules, 193(A): p. 137-144).

Throughout the disclosure and claims, a “mammalian milk oligosaccharide (MMO)” is preferably a “human milk oligosaccharide (HMO).”

In an additional and/or alternative more preferred embodiment, the sialylated saccharide according to this disclosure comprises a lactose, a lacto-N-biose (LNB) or N-acetyllactosamine (LacNAc) at its reducing end, preferably the sialylated saccharide comprises lactose or LacNAc at its reducing end, most preferably the sialylated saccharide comprises lactose at its reducing end.

A sialylated saccharide comprising lactose at its reducing end is preferably selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c and LST d; more preferably selected from the list consisting of 3′SL, 6′SL, 3,6-disialyllactose, 3'S-2′FL, 6'S-2′FL, 3'S-3-FL, 6'S-3-FL, LST a, LST b, LST c and LST d; even more preferably selected from the list consisting of 3′SL, 6′SL, 3'S-2′FL, 6'S-2′FL, 3'S-3-FL, 6'S-3-FL, LST a, LST b, LST c and LST d; even more preferably selected from 3′SL, 3'S-2′FL, 3'S-3-FL, LST a and LST d, even more preferably 3′SL, LST a or LST d, most preferably 3′SL.

A sialylated saccharide comprising lacto-N-biose (LNB) at its reducing end is preferably selected form the list consisting of 3′-sialyllacto-N-biose (3′SLNB), 6′-sialyllacto-N-biose (6′SLNB) and sialyl Lewis a; more preferably the sialylated saccharide is 3′SLNB or sialyl Lewis a; most preferably 3′SLNB.

A sialylated saccharide comprising N-acetyllactosamine (LacNac) at its reducing end is preferably selected form the list consisting of 3′-sialyllactosamine (3′SLacNAc), 6′-sialyllactosamine (6′SLacNAc) and sialyl Lewis x; more preferably the sialylated saccharide is 3′SLacNAc or sialyl Lewis x; most preferably 3′SLacNAc.

In an even more preferred embodiment, the sialylated saccharide according to this disclosure is selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; more preferably selected from the list consisting of 3′-sialyllactose (3′SL), 6′-sialyllactose (6′SL), 3,6-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; even more preferably selected from the list consisting of 3′-sialyllactose (3′SL), 6′-sialyllactose (6′SL), 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; even more preferably selected form the list consisting of 3′-sialyllactose (3′SL), 6′-sialyllactose (6′SL), LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; even more preferably selected from the list consisting of 3′-sialyllactose (3′SL), 6′-sialyllactose (6′SL), 3′-sialyllacto-N-biose (3′SLNB), 6′-sialyllacto-N-biose (6′SLNB), 3′-sialyllactosamine (3′SLacNAc) and 6′-sialyllactosamine (6′SLacNAc); even more preferably selected from the list consisting of 3′-sialyllactose (3′SL), 3′-sialyllacto-N-biose (3′SLNB) and 3′-sialyllactosamine (3′SLacNAc), most preferably 3′-sialyllactose (3′SL).

In the context of this disclosure, a sialylated saccharide comprising a sialic acid, preferably Neu5Ac, linked to a monosaccharide through an alpha-2,3-linkage is particularly advantageous in preventing/reducing an inflammatory disease and/or autoimmune disease as described herein, preferably wherein the monosaccharide is preferably selected from galactose, N-acetylglucosamine and Neu5Ac, more preferably the monosaccharide is galactose or N-acetylglucosamine, even more preferably the monosaccharide is galactose. Such a sialylated saccharide is preferably selected from the list consisting of 3′-sialyllactose (3′SL), 3,6-disialyllactose, 8,3-disialyllactose, 3'S-2′FL, 3'S-3-FL, LST a, LST d, 3′-sialyllacto-N-biose (3′SLNB), sialyl Lewis a, 3′-sialyllactosamine (3′SLacNAc), and sialyl Lewis x; more preferably selected from the list consisting of 3′-sialyllactose (3′SL), 3'S-2′FL, 3'S-3-FL, LST a, LST d, 3′-sialyllacto-N-biose (3′SLNB), sialyl Lewis a, 3′-sialyllactosamine (3′SLacNAc), and sialyl Lewis x. It is preferred that such a sialylated saccharide comprises lactose or LacNAc at its reducing end, most preferably such a sialylated saccharide comprises lactose at its reducing end.

As elaborated earlier herein, in some embodiments of this disclosure, two or more sialylated saccharides (i.e., a first sialylated saccharide and a second sialylated saccharide, optionally further sialylated saccharides are present), preferably two sialylated saccharides (i.e., a first sialylated saccharide and a second sialylated saccharide), are administered to the subject. For the sake of clarity, the expression “preferably two sialylated saccharides” in this context of this disclosure means that no additional sialylated saccharides are administered to the subject.

All the embodiments relating to “a sialylated saccharide” of the Section “Saccharide” apply to “first sialylated saccharide” as described in the disclosure and claims (i.e., according to this disclosure), unless specifically stated otherwise. In other words, each embodiment disclosed in the context of “a sialylated saccharide” is considered to be explicitly disclosed in the context of “a first sialylated saccharide,” unless specifically stated otherwise.

It is a preferred embodiment that the second sialylated saccharide is as described earlier herein in the context of a sialylated saccharide. In other words, each embodiment disclosed in the context of “a sialylated saccharide” is considered to be explicitly disclosed in the context of “a second sialylated saccharide,” unless specifically stated otherwise.

Alternatively, it is a preferred embodiment that the second sialylated saccharide comprises a sialic acid, preferably Neu5Ac, that is linked to a monosaccharide in an alpha-2,3-, alpha-2,6- or alpha-2,8-linkage, preferably an alpha-2,6- or an alpha-2,8-linkage, more preferably an alpha-2,6-linkage, and wherein the monosaccharide is preferably selected from galactose, N-acetylglucosamine and Neu5Ac, more preferably the monosaccharide is galactose or N-acetylglucosamine, even more preferably the monosaccharide is galactose. It is further preferred that the second sialylated saccharide is a disaccharide or an oligosaccharide, more preferably an oligosaccharide (preferably as defined earlier herein). More preferably, the oligosaccharide consists of 3-9, preferably 3-8, more preferably 3-7, even more preferably 3-6, most preferably 3-5, monosaccharides.

In an additional and/or alternative preferred embodiment, the second sialylated saccharide according to this disclosure is a milk saccharide and/or a Lewis-type antigen saccharide.

Preferably, the Lewis-type antigen saccharide is sialyl Lewis a or sialyl Lewis x.

In a more preferred embodiment, the second sialylated saccharide according to this disclosure is a milk saccharide, preferably a milk oligosaccharide. Preferably, the milk saccharide/milk oligosaccharide is a mammalian milk saccharide/mammalian milk oligosaccharide (MMO). More preferably, the milk saccharide/milk oligosaccharide is a human milk saccharide/human milk oligosaccharide (HMO).

In an additional and/or alternative more preferred embodiment, the second sialylated saccharide according to this disclosure comprises a lactose, a lacto-N-biose (LNB) or N-acetyllactosamine (LacNAc) at its reducing end, preferably the second sialylated saccharide comprises lactose or LacNAc at its reducing end, most preferably the second sialylated saccharide comprises lactose at its reducing end.

A second sialylated saccharide comprising lactose at its reducing end is preferably selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c and LST d; more preferably selected from the list consisting of 3′SL, 6′SL, 6,6-disialyllactose, 3'S-2′FL, 6'S-2′FL, 3'S-3-FL, 6'S-3-FL, LST a, LST b, LST c and LST d; even more preferably selected from the list consisting of 3′SL, 6′SL, 3'S-2′FL, 6'S-2′FL, 3'S-3-FL, 6'S-3-FL, LST a, LST b, LST c and LST d; even more preferably selected from 6′SL, 6'S-2′FL, 6'S-3-FL, LST b and LST c, even more preferably 6′SL, LST b or LST c, most preferably 6′SL.

A second sialylated saccharide comprising lacto-N-biose (LNB) at its reducing end is preferably selected form the list consisting of 3′-sialyllacto-N-biose (3′SLNB), 6′-sialyllacto-N-biose (6′SLNB) and sialyl Lewis a; more preferably the second sialylated saccharide is 6′SLNB or sialyl Lewis a; most preferably 6′SLNB.

A second sialylated saccharide comprising N-acetyllactosamine (LacNac) at its reducing end is preferably selected form the list consisting of 3′-sialyllactosamine (3′SLacNAc), 6′-sialyllactosamine (6′SLacNAc) and sialyl Lewis x; more preferably the second sialylated saccharide is 6′SLacNAc or sialyl Lewis x; most preferably 6′SLacNAc.

In an even more preferred embodiment, the second sialylated saccharide according to this disclosure is selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; more preferably selected from the list consisting of 3′-sialyllactose (3′SL), 6′-sialyllactose (6′SL), 6,6-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; even more preferably selected from the list consisting of 3′-sialyllactose (3′SL), 6′-sialyllactose (6′SL), 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; even more preferably selected form the list consisting of 3′-sialyllactose (3′SL), 6′-sialyllactose (6′SL), LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; even more preferably selected from the list consisting of 3′-sialyllactose (3′SL), 6′-sialyllactose (6′SL), 3′-sialyllacto-N-biose (3′SLNB), 6′-sialyllacto-N-biose (6′SLNB), 3′-sialyllactosamine (3′SLacNAc) and 6′-sialyllactosamine (6′SLacNAc); even more preferably selected from the list consisting of 6′-sialyllactose (6′SL), 6′-sialyllacto-N-biose (6′SLNB) and 6′-sialyllactosamine (6′SLacNAc), most preferably 6′-sialyllactose (6′SL).

In an even more preferred embodiment, the second sialylated saccharide according to this disclosure comprises a sialic acid, preferably Neu5Ac, that is linked to a monosaccharide in an alpha-2,6-linkage as described herein and is selected from the list consisting of 6′-sialyllactose (6′SL), 3,6-disialyllactose, 6,6-disialyllactose, 6'S-2′FL, 6'S-3-FL, LST b, LST c, 6′-sialyllacto-N-biose (6′SLNB) and 6′-sialyllactosamine (6′SLacNAc); more preferably selected from the list consisting of 6′-sialyllactose (6′SL), 6,6-disialyllactose, 6'S-2′FL, 6'S-3-FL, LST b, LST c, 6′-sialyllacto-N-biose (6′SLNB) and 6′-sialyllactosamine (6′SLacNAc); even more preferably selected form the list consisting of 6′-sialyllactose (6′SL), 6'S-2′FL, 6'S-3-FL, LST b, LST c, 6′-sialyllacto-N-biose (6′SLNB) and 6′-sialyllactosamine (6′SLacNAc); even more preferably selected from the list consisting of 6′-sialyllactose (6′SL), 6'S-2′FL, 6'S-3-FL, LST b, LST c and 6′-sialyllactosamine (6′SLacNAc); even more preferably selected from the list consisting of 6′-sialyllactose (6′SL), 6'S-2′FL, 6'S-3-FL, LST b and LST c, most preferably 6′-sialyllactose (6′SL). It is preferred that the second sialylated saccharide comprises lactose or LacNAc at its reducing end, most preferably that the second sialylated saccharide comprises lactose at its reducing end.

Aforementioned sialylated saccharides are commercially available and/or the production/purification of these saccharides has been described and hence allows the skilled person to produce/obtain any of the sialylated saccharides accordingly. For example (each reference is incorporated by reference):
3′SL: Zhang et al, 2022, ACS Synth. Biol. 11(8): p. 2837-2845; Carbosynth (OS04397)
6′SL: Guo et al, 2018, Appl. Environ. Microbiol. 84(13): e00071-18; Carbosynth (OS04398)
3,6-disialyllactose: Pan et al, 2006, Carbohydr. Res. 341(6): p. 730-737
6,6′-disialyllactose: Drouillard et al, 2010, Carbohydr. Res. 345(10): p. 1394-1399
8,3-disialyllactose: Carbosynth (OD45739)
3'S-3-FL: Biosynth/Cymit Quimica (3D-OSO1065)
LST a: Carbosynth (OL03882)
LST b: Carbosynth (OL03877)
LST c: WO2016/199071; Carbosynth (OL06570)
LST d: Carbosynth (OS158776)
3′SLNB: Carbosynth (OS35289)
Sialyl lewis a: Carbosynth (OS00745)
Sialyl lewis x: Yu et al, 2017, Chem. Commun (Camb) 53(80): p. 11012-11015; Carbosynth (OS04058)

In a preferred embodiment, the sialylated saccharide according to this disclosure has been isolated from a microbial cultivation or fermentation, cell culture, enzymatic reaction or chemical reaction. Alternatively, the sialylated saccharide of this disclosure has been isolated by e.g., chromatography or filtration technology from a natural source such as a human or animal milk, preferably animal milk. Isolation of the sialylated saccharide typically results in a solution containing the saccharide. Such a solution can, for example, be obtained by a method comprising the steps of:
(a) cultivating at least one cell as defined herein, preferably a single cell, that is capable to produce the sialylated saccharide, in a suitable cultivation medium to form a cultivation broth, preferably wherein the cell is metabolically engineered for the production of the sialylated saccharide, and
(b) purifying the sialylated saccharide, from the cultivation broth by:
   (i) clarifying the cultivation broth, and
   (ii) removing salts and/or medium components form the clarified cultivation broth, and/or
   (iii) concentrating the saccharide in the clarified cultivation broth,
thereby providing a solution comprising a purified sialylated saccharide.

In the context of this disclosure, “clarifying the cultivation broth” refers to the removal of suspended particulates and contaminants, particularly cells, cell components, insoluble metabolites and debris produced by culturing the cell according to this disclosure. Clarification is preferably one or more of centrifugation, flocculation, decantation and/or filtration.

In the context of this disclosure, “removing salts and/or medium components form the clarified cultivation broth” refers to removing substantially all the proteins, as well as peptides, amino acids, RNA and DNA and any endotoxins and glycolipids from the clarified cultivation broth, preferably after it has been. In this step, proteins and related impurities can be removed from the saccharide in a conventional manner. Preferably, proteins, salts, by-products, color, endotoxins and other related impurities are removed from the saccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including e.g., DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, the oligosaccharide remains in the oligosaccharide containing clarified cultivation broth.

Further purification of the saccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product.

Several drying techniques are known to the skilled person that can be used to obtain powder from a solution containing the sialylated saccharide. Powder is preferably obtained by spray drying, freeze drying, spray freeze-drying, crystallization, lyophilization, band or belt drying, drum or roller drying, and/or agitated thin film drying, preferably by spray drying, drum or roller drying, or agitated thin film drying, more preferably by spray drying or agitated thin film drying, most preferably by spray drying, of a solution containing the sialylated saccharide Preferably, the sialylated saccharide according to this disclosure constitutes ≥70.0%, preferably ≥75.0%, more preferably ≥80.0, (w/w) of the powder. Preferably, the powder contains ≤15 wt. %, preferably ≤10 wt. %, more preferably ≤9 wt. %, more preferably ≤8 wt. %, more preferably ≤7 wt. %, even more preferably ≤5 wt. %, even more preferably ≤4 wt. % of liquid, even more preferably ≤3 wt. % of liquid, even more preferably ≤2 wt. % of liquid, most preferably ≤1 wt. %, preferably wherein the liquid is water.

In an additional and/or alternative preferred embodiment, the sialylated saccharide according to this disclosure has been produced, preferably in vitro and/or ex vivo, by a cell, preferably a single cell, wherein the cell is preferably chosen from the list consisting of a microorganism, a plant cell, an animal cell and a protozoan cell. In other words, the sialylated saccharide of this disclosure has been produced by an in vitro and/or ex vivo culture of cells, wherein the cells are preferably chosen from the list consisting of a microorganism, a plant cell, an animal cell or a protozoan cell.

Preferably, the cell is a microorganism. Preferably, the microorganism is selected from a list consisting of a bacterium, a yeast and a fungus. Further, it is preferred that the cell is genetically engineered for the production of the sialylated saccharide according to this disclosure. Throughout the description and claims, the terms “has been isolated” and “is obtained” are interchangeably used, unless specifically stated otherwise.

The latter bacterium preferably belongs to the phylum of the Proteobacteria or the phylum of the Firmicutes or the phylum of the Cyanobacteria or the phylum Deinococcus-Thermus, preferably belongs to the phylum of the Proteobacteria. The latter bacterium belonging to the phylum Proteobacteria belongs preferably to the family Enterobacteriaceae, preferably to the species Escherichia coli. The latter bacterium preferably relates to any strain belonging to the species Escherichia co/i such as but not limited to Escherichia coli B, Escherichia coli C, Escherichia coli W, Escherichia coli K12, Escherichia coli Nissle. More specifically, the latter term relates to cultivated E. coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably this disclosure specifically relates to a mutated and/or transformed Escherichia coli strain as indicated above wherein the E. coli strain is a K12 strain. More specifically, this disclosure relates to a mutated and/or transformed Escherichia coli strain as indicated above wherein the K12 strain is E. coli MG1655. The latter bacterium belonging to the phylum Firmicutes belongs preferably to the Bacilli, preferably from the species Bacillus, such as Bacillus subtilis or B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g., Saccharomyces cerevisiae, S. bayanus, S. boulardii), Pichia (with members like e.g., Pichia pastoris, P. anomala, P. kluyveri), Komagataella, Hansunella, Kluyveromyces (with members like e.g., Kluyveromyces lactis, K. marxianus, K. thermotolerans), Yarrowia (like e.g., Yarrowia lipolytica), Eremothecium, Zygosaccharomyces, Starmerella (like e.g., Starmerella bombicola) or Debaromyces. The latter yeast is preferably selected from Pichia pastoris, Yarrowia lipolitica, Saccharomyces cerevisiae and Kluyveromyces lactis. The latter fungus belongs preferably to the genus Rhizopus, Dictyostelium, Penicillium, Mucor or Aspergillus. “Plant cells” includes cells of flowering and non-flowering plants, as well as algal cells, for example, Chlamydomonas, Chlorella, etc. Preferably, the plant cell is a tobacco, alfalfa, rice, cotton, rapeseed, tomato, corn, maize or soybean cell. The latter animal cell is preferably derived from non-human mammals (e.g., cattle, buffalo, pig, sheep, mouse, rat), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the list comprising an epithelial cell like e.g., a mammary epithelial cell, mammary myoepithelial cell, mammary progenitor cell, an embryonic kidney cell (e.g., HEK293 or HEK 293T cell), a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO21067641, preferably mesenchymal stem cell or derivates thereof as described in WO21067641. The insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

In an additional and/or alternative preferred embodiment, the sialylated saccharide of this disclosure has been produced, preferably in vitro and/or ex vivo, by a mammary epithelial cell, mammary myoepithelial cell and/or mammary progenitor cell, preferably wherein the cell is generated from non-mammary adult stem cells, more preferably wherein the cell is generated from mesenchymal stem cells.

Such cells are well-known to the skilled person, it is in this regard referred to in, for example, WO2021/067641 and WO2021/242866 (mammary epithelial cells derived from non-mammary adult stem cells, preferably from mesenchymal stem cells) and WO2021/142241 (mammary epithelial cells, mammary myoepithelial cells, mammary progenitor cells).

In a more preferred embodiment, the sialylated saccharide of this disclosure has been produced, preferably in vitro and/or ex vivo, by a microorganism cell, preferably the microorganism is a bacterium, a yeast or a fungus, more preferably the microorganism is a bacterium or a yeast, even more preferably the microorganism is a bacterium, most preferably the microorganism is Escherichia coli.

In the context of this disclosure, it is also within the scope of this disclosure that two or more different cells (preferably as defined herein), produce the sialylated saccharide according to this disclosure. It is however preferred that a single cell produces the sialylated saccharide, i.e., a single culture of the cell produces the sialylated saccharide of this disclosure.

In another preferred embodiment of this disclosure, the sialylated saccharide, and optionally any, preferably all, further sialylated saccharides, is linked, preferably chemically linked, to a carrier for delivery of the saccharide(s). The linking of the sialylated saccharide to the carrier increases the avidity.

Preferably, the carrier is a ceramide-based carrier or a polypeptide-based carrier, more preferably the carrier is a ceramide-based carrier. Preferably, the polypeptide-based carrier is epsilon-polylysine, alfa-polylysine, poly(aspartic acid), polyglutamic acid or polyornithine. These carriers are commercially available (e.g., Sigma-Aldrich, Carbosynth). The ceramide-based carrier is preferably selected from a list consisting of d18:1/16:0, t18:0-16:0, t18:0-h16:0, t18:0-h22:0 and t18:0-h24:0. These ceramide carriers are commercially available and well-known to the skilled person and are, for example, described in WO2010/037785, which is incorporated by reference. d18:1/16:0 is also known as C16 ceramide and N-palmitoylsphingosine and therefore interchangeably used herein. t18:0-16:0 is also known as C16 phytoceramide and N-hexadecanoyl phytosphingosine and therefore interchangeable used herein. t18:0-h16:0, t18:0-h22:0 and t18:0-h24:0 are glycosylinositolphosphoceramides (GIPCs). As known to the skilled person, “d” and “t” refer to the hydroxylation state of the whole ceramide or long-chain base moiety (d is 2 groups, t is 3 groups), whereas “h” denotes a hydroxylation of the fatty acyl group.

The sialylated saccharide and optionally any, preferably all, further sialylated saccharides, is preferably encapsulated (i.e., forming an encapsulate). This is particularly advantageous to protect the saccharide(s) from conditions relating to the manufacturing or storing of a composition as described in the Section “Composition,” especially when the composition is a nutritional composition (it is referred to in the Section “Composition”). Such conditions typically entail a high pressure (e.g., a pressure of above 10 bar during extrusion), a high temperature (e.g., a temperature above 100° C. during extrusion and baking), a pH lower than 5, a pH higher than 8, and/or a high moisture content (e.g., wet extrusion). Moreover, other molecules within a composition, especially a nutritional composition, such as proteins could chemically interact with the sialylated saccharide (and any other further saccharide if present) especially under the harsh conditions during manufacturing of a nutritional composition. Hence, throughout the disclosure and claims, the “sialylated saccharide” is preferably replaced with “encapsulated sialylated saccharide.” If any further saccharide, preferably milk saccharide, is present than it is preferred that all milk saccharides, preferably all saccharides, present in the composition are encapsulated, preferably all milk saccharides, preferably all saccharides, are co-encapsulated.

The encapsulation is particularly advantageous when the composition according to this disclosure is a nutritional composition (it is referred to in the Section “Composition”) that is manufactured by extrusion (dry or wet, preferably wet).

It is preferred that the encapsulate is of the core-shell type, i.e., carrier material forms a shell around the active agent (i.e., the sialylated saccharide according to this disclosure), preferably wherein the core has a median diameter (D(v,0.5)) of 125-250 μm, preferably 150-250 μm, more preferably 165-250 μm, most preferably 165-225 μm, and wherein the encapsulate has a median diameter (D(v,0.5)) that is 20-100 μm, preferably 20-75 μm, more preferably 35-75 μm, longer than the median diameter of the core.

The carrier material preferably comprises a hot melt material, more preferably an oil and/or a wax, even more preferably a hydrogenated oil and/or a wax, optionally further comprising starch, preferably wherein the starch constitutes less than 15% (w/w) of the carrier material, more preferably the starch constitutes less than 10% (w/w) of the carrier material.

The oil is preferably selected from the list consisting of palm oil, sunflower oil, soybean oil, rapeseed oil, coconut oil, babassu oil, palm kernel oil, maize oil, sesame oil and cottonseed oil; more preferably the oil is selected from the list consisting of palm oil, sunflower oil, soybean oil and rapeseed oil; even more preferably the oil is palm oil, most preferably the oil is hydrogenated palm oil.

The wax is preferably selected from the list consisting of Candelilla wax, Carnauba wax, beeswax, rice bran wax, paraffin wax, jojoba wax, microcrystalline wax and japan wax; more preferably the wax is Candelilla wax or Carnauba wax; most preferably the wax is Candelilla wax.

It is further preferred that the sialylated saccharide or all sialylated saccharides or all milk saccharides present in the encapsulate constitute 5-50% (w/w), preferably 10-40% (w/w), more preferably 20-40% (w/w), even more preferably 20-35% (w/w), of the total weight of the encapsulate.

Further, it is a preferred embodiment that the sialylated saccharide according to this disclosure is part of a composition, preferably a composition as described in the Section “Composition.”

In an additional and/or alternative preferred embodiment, the first sialylated saccharide according to this disclosure is part of a composition, preferably a composition as described in the Section “Composition.”

In an additional and/or alternative preferred embodiment, the second sialylated saccharide according to this disclosure is part of a composition, preferably a composition as described in the Section “Composition.”

In the context of this disclosure, each saccharide according to this disclosure that is administered to the subject according to this disclosure, are preferably part of the same composition, preferably a composition as described in the Section “Composition.”

### Composition

In an embodiment of the first aspect of this disclosure, the sialylated saccharide is for use in a method for preventing and/or treating an inflammatory disease and/or autoimmune disease in a subject.

In a preferred embodiment, the sialylated saccharide according to this disclosure is part of a composition, preferably a composition as described in the Section “Composition.” In other words, the sialylated saccharide is for use in a method for preventing and/or treating an inflammatory disease and/or autoimmune disease in a subject, wherein the sialylated saccharide is part of a composition, preferably a composition as described in the Section “Composition.”

In an additional and/or alternative preferred embodiment, the first sialylated saccharide according to this disclosure is part of a composition, preferably a composition as described in the Section “Composition.” In other words, the first sialylated saccharide is for use in a method for preventing and/or treating an inflammatory disease and/or autoimmune disease in a subject, wherein the first sialylated saccharide is part of a composition, preferably a composition as described in the Section “Composition.”

In an additional and/or alternative preferred embodiment, the second sialylated saccharide according to this disclosure is part of a composition, preferably a composition as described in the Section “Composition.” In other words, the second sialylated saccharide is for use in a method for preventing and/or treating an inflammatory disease and/or autoimmune disease in a subject, wherein the second sialylated saccharide is part of a composition, preferably a composition as described in the Section “Composition.”

In the context of this disclosure, each saccharide according to this disclosure that is administered to the subject according to this disclosure, are preferably part of the same composition, preferably a composition as described in the Section “Composition.” As the skilled person knows in the context of a medical treatment/prevention, it is equivalent to providing a subject with either one composition comprising two compounds or two separate compositions, each composition comprising a single compound. What matters is that the subject ultimately receives both compounds.

In a more preferred embodiment, this disclosure provides a composition for use in a method for preventing and/or treating an inflammatory disease and/or autoimmune disease in a subject, wherein the composition comprises a sialylated saccharide. Preferably, wherein the composition is a composition as described in the Section “Composition.”

The “subject” is as described in the Section “Subject.” The “method for preventing and/or treating” is as described in the Section “Method for preventing and/or treating.” The “inflammatory disease” and the “autoimmune disease” are as described in the Section “Inflammatory disease/autoimmune disease.” The “sialylated saccharide” is as described in the Section “Saccharide.” For the sake of clarity, the Section “Mode of Action” also applies in this context of the disclosure.

As elaborated in the Section “Saccharide,” it is preferred that two or more sialylated saccharides (i.e., a first sialylated saccharide and a second sialylated saccharide, optionally further sialylated saccharides are present), preferably two sialylated saccharides (i.e., a first sialylated saccharide and a second sialylated saccharide), are administered to the subject. Hence, this disclosure preferably provides a composition for use in a method for preventing and/or treating an inflammatory disease and/or autoimmune disease in a subject, wherein the composition comprises a first sialylated saccharide and a second sialylated saccharide.

The “subject” is as described in the Section “Subject.” The “method for preventing and/or treating” is as described in the Section “Method for preventing and/or treating.” The “inflammatory disease” and the “autoimmune disease” are as described in the Section “Inflammatory disease/autoimmune disease.” The “first sialylated saccharide” and the “second sialylated saccharide” are as described in the Section “Saccharide.” For the sake of clarity, the Section “Mode of Action” also applies in this context of the disclosure.

In a preferred embodiment of this disclosure, the composition according to this disclosure is not mother's milk.

In an additional and/or alternative preferred embodiment, the composition according to this disclosure is a synthetic composition. Throughout the disclosure and claims, the term “composition” is preferably replaced with “synthetic composition,” unless specifically stated otherwise.

In the context of this disclosure, a “synthetic composition” or a “synthetic nutritional composition” or a “synthetic pharmaceutical composition” refers to a composition that is artificially prepared and preferably refers to a composition comprising at least one component that is produced ex vivo, either chemically and/or biologically, e.g., by means of chemical reaction, enzymatic reaction or recombinantly, or purified by humans. It is preferred that a synthetic composition of this disclosure is not identical with a naturally occurring composition.

In this context, it is preferred that the sialylated saccharide according to this disclosure (it is referred to in the Section “Saccharide”) has been isolated from a microbial cultivation or fermentation, cell culture, enzymatic reaction or chemical reaction; or has been isolated from a natural source (it is referred to in the Section “Saccharide”). It is more preferred that the sialylated saccharide has been produced in vitro and/or ex vivo by a cell (it is referred to in the Section “Oligosaccharide”).

In an additional and/or alternative preferred embodiment, the composition according to this disclosure further comprises one or more probiotics (i.e., microbial strains) for its beneficial effect on the subject's gut microbiome, preferably a microbial strain that does not use, preferably is not able to use, the sialylated saccharide as a carbon source. Examples of probiotics include Bifidobacterium, Lactobacillus and Saccharomyces boulardii.

In an alternative preferred embodiment, the composition according to this disclosure does not comprise a microbial strain. Examples of such a strain are Bifidobacterium, Lactobacillus and Saccharomyces boulardii.

In a more preferred embodiment, the composition according to this disclosure is a pharmaceutical composition, optionally further comprising a pharmaceutically acceptable carrier, filler, preservative, solubilizer, diluent, surfactant, excipient, salt, adjuvant and/or solvent.

The term “pharmaceutically acceptable” components refers to a component that does not cause significant toxic effects on the subject and does not interfere with the biological activity and properties of the sialylated saccharide according to this disclosure.

The use of a carrier, filler, preservative, solubilizer, diluent, surfactant, excipient, salt, adjuvant and solvent in pharmaceutical compositions is well-known to the skilled person. Reference may be made to Remington: the science and practice of pharmacy, 23rd edition, 2020.

The term “carrier” refers to a substance that can be added to the sialylated saccharide according to this disclosure (i.e., the active ingredient) to help formulating or stabilizing the formulation.

Suitable acids for the preparation of a pharmaceutically acceptable salt include acetic acid, 2, 2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(IS)-camphor-10-sulfonic acid, capric acid, caproic acid, caprylic acid, cinnamic acid, citric acid, cyclamic acid, cyclohexanesulfamic acid, dodecylsulfuric acid, ethane-1, 2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, -oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1, 5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid and valeric acid.

Suitable bases for the preparation of a pharmaceutically acceptable salt include inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1, 3-propanediol, and tromethamine.

Suitable diluents include dicalcium phosphate, calcium sulfate, lactose, sorbitol, sucrose, inositol, cellulose, kaolin, mannitol, sodium chloride, dry starch and powdered sugar.

Suitable preservatives include glycerin, methyl and propylparaben, benzoic add, sodium benzoate and alcohol.

In this context of this disclosure, the sialylated saccharide according to this disclosure may also be designed as a prodrug, i.e., a functional derivative of the sialylated saccharide that is readily convertible into the actual sialylated saccharide (i.e., so-called parent saccharide or parent drug) in vivo. Prodrugs are often useful because, in some situations, they may be easier to administer than the parent saccharide. A prodrug may be converted into the parent drug by various mechanisms, including enzymatic processes and metabolic hydrolysis as known in the art.

A pharmaceutical composition according to this disclosure may be formulated in a solid, semisolid or liquid dosage form, preferably for oral administration. As used herein, oral administration also includes buccal, lingual, and sublingual administration. Suitable oral dosage forms include, but are not limited to, tablets, capsules, pills, troches, lozenges, pastimes, cachets, pellets, medicated chewing gum, gummy, candy, granules, bulk powders, effervescent or non-effervescent powders or granules, solutions, emulsions, suspensions, solutions, wafers, sprinkles, elixirs and syrups.

The term “dosage form” as understood by the skilled person refers to the physical form of a dose of the sialylated saccharide according to this disclosure that is used as a drug or medication intended for administration or consumption.

A pharmaceutical composition according to this disclosure may be formulated as an immediate or modified release dosage form, including delayed-, sustained, pulsed-, controlled-, targeted- and programmed-release dosage form. The term “modified release dosage form” refers to a dosage form in which the rate and/or place of release of the active agent (i.e., the sialylated saccharide according to this disclosure) is different from that of an immediate dosage form when administer by the same route (e.g., oral route). Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient (i.e., sialylated saccharide according to this disclosure) can also be modified by varying the particle sizes and polymorphism of the active ingredient(s).

In an additional and/or alternative more preferred embodiment, the composition is a nutritional composition, optionally further comprising a feed ingredient and/or a food ingredient, wherein the feed/food ingredient is preferably chosen from the list consisting of:
a lipid, preferably one or more selected from the list consisting of an oil, fat, ester, monoglyceride, diglyceride, triglyceride and free fatty acid;
a vitamin, preferably one or more selected from the list consisting of vitamin A, vitamin B, vitamin C, vitamin D, vitamin E and vitamin H, or a derivate thereof,
an amino acid compound;
a trace element;
a mineral, preferably one or more selected from the list consisting of calcium, phosphorus, magnesium, iron, zinc, manganese, copper, sodium, potassium, molybdenum, chromium, selenium and chloride;
an antioxidant;
a prebiotic agent, preferably one or more selected from the list consisting of GOS (galacto-oligosaccharide), FOS (fructo-oligosaccharide), inulin and resistant starch;
a carbohydrate;
an antimicrobial agent; and/or
a protein.

Generally, any source of protein may be used so long as it is suitable for nutritional compositions and is otherwise compatible with any other selected ingredients or features in the nutritional composition. Non-limiting examples of suitable proteins (and sources thereof) suitable for use in the nutritional composition according to this disclosure include, but are not limited to, intact, hydrolyzed, or partially hydrolyzed protein, which may be derived from any known or otherwise suitable source such as milk (e.g., casein, whey), animal (e.g., meat, fish), cereal (e.g., rice, corn, wheat), vegetable (e.g., soy, pea, potato, bean), and combinations thereof. The protein may also include a mixture of amino acids (often described as free amino acids) known for use in nutritional products or a combination of such amino acids with the intact, hydrolyzed, or partially hydrolyzed proteins described herein. The amino acids may be naturally occurring or synthetic amino acids. More particular examples of suitable protein (or sources thereof) used in a nutritional composition according to this disclosure include, but are not limited to, whole cow's milk, partially or completely defatted milk, milk protein concentrates, milk protein isolates, nonfat dry milk, condensed skim milk, whey protein concentrates, whey protein isolates, acid caseins, sodium caseinates, calcium caseinates, potassium caseinates, legume protein, soy protein concentrates, soy protein isolates, pea protein concentrates, pea protein isolates, collagen proteins, potato proteins, rice proteins, wheat proteins, canola proteins, quinoa, insect proteins, earthworm proteins, fungal (e.g., mushroom) proteins, hydrolyzed yeast, gelatin, bovine colostrum, human colostrum, glycol macropeptides, mycoproteins, proteins expressed by microorganisms (e.g., bacteria and algae), and combinations thereof. A nutritional composition according to this disclosure may include any individual source of protein or combination of the various sources of protein listed above. In addition, the proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

The carbohydrate or source of carbohydrate suitable for use in a nutritional composition according to this disclosure may be simple, complex, or variations or combinations thereof. Generally, the carbohydrate may include any carbohydrate or carbohydrate source that is suitable for use nutritional compositions and is otherwise compatible with any other selected ingredients or features in the nutritional composition. Non-limiting examples of carbohydrates suitable for use in the nutritional composition according to this disclosure, but are not limited to, polydextrose, maltodextrin; hydrolyzed or modified starch or cornstarch; glucose polymers; corn syrup; corn syrup solids; sucrose; glucose; fructose; lactose; high fructose corn syrup; honey; sugar alcohols (e.g., maltitol, erythritol, sorbitol); isomaltulose; sucromalt; pullulan; potato starch; and other slowly-digested carbohydrates; dietary fibers including, but not limited to, fructooligosaccharides (FOS), galactooligosaccharides (GOS), oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinogalactans, glucomannan, xanthan gum, alginate, pectin, low methoxy pectin, high methoxy pectin, cereal beta-glucans (e.g., oat beta-glucan, barley beta-glucan), carrageenan and psyllium, soluble and insoluble fibers derived from fruits or vegetables; other resistant starches; and combinations thereof. A nutritional composition according to this disclosure may include any individual source of carbohydrate or combination of the various sources of carbohydrate listed above.

The fat or source of fat suitable for use in a nutritional composition according to this disclosure may be derived from various sources including, but not limited to, plants, animals, and combinations thereof. Generally, the fat may include any fat or fat source that is suitable for use in a nutritional composition according to this disclosure and is otherwise compatible with any other selected ingredients or features in the nutritional composition. Non-limiting examples of suitable fat (or sources thereof) for use in a nutritional composition according to this disclosure include coconut oil, fractionated coconut oil, soy oil, high oleic soy oil, corn oil, olive oil, safflower oil, high oleic safflower oil, medium chain triglyceride oil (MCT oil), high gamma linolenic (GLA) safflower oil, sunflower oil, high oleic sunflower oil, palm oil, palm kernel oil, palm olein, canola oil, high oleic canola oil, marine oils, fish oils, algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, rice bran oil, wheat bran oil, interesterified oils, transesterified oils, structured lipids, and combinations thereof. Generally, the fats used in a nutritional composition for formulating infant formulas and pediatric formulas provide fatty acids needed both as an energy source and for the healthy development of the infant, toddler, or child. These fats typically comprise triglycerides, although the fats may also comprise diglycerides, monoglycerides, and free fatty acids. Fatty acids provided by the fats in the nutritional composition include, but are not limited to, capric acid, lauric acid, myristic acid, palmitic acid, palmitoleic acid, stearic acid, oleic acid, linoleic acid, alpha-linolenic acid, ARA, EPA, and DHA. The nutritional composition can include any individual source of fat or combination of the various sources of fat listed above. Preferably, the fat is a mixture of vegetable fat and milk fat such as obtained from milk from a mammal like cow, sheep, goat, mare, or camel. More preferably, wherein the milk fat is bovine milk fat. Mixtures of different types of fat are preferred because they help to provide different fatty acids and better resemble the type of linkage between the glycerol moiety and the fatty acid moiety in the fat, when compared to human mother's milk.

When one or more prebiotics is/are present, then it is preferred that the weight ratio between the sialylated saccharide according to this disclosure and the prebiotic(s) is in the range of from 0.5:10 to 10:0.5.

It has been found that a sialylated saccharide (most preferably a first sialylated saccharide and a second sialylated saccharide) according to this disclosure (it is referred to in the Section “Saccharide”) is sufficient to obtain an effective therapeutic for preventing and/or treating an inflammatory disease or an autoimmune disease as described herein. Administering of additional saccharide(s) is not required to obtain the therapeutic effect.

In this context, it is hence a preferred embodiment that the composition according to this disclosure does not comprise lacto-N-tetraose (LNT). Likewise, it is a preferred embodiment that the method for preventing and/or treating an inflammatory disease or an autoimmune disease according to this disclosure does not comprise the administration of lacto-N-tetraose (LNT).

In an additional and/or alternative preferred embodiment, the composition according to this disclosure does not comprise lacto-N-neotetraose (LNnT). Likewise, it is a preferred embodiment that the method for preventing and/or treating an inflammatory disease or an autoimmune disease according to this disclosure does not comprise the administration of lacto-N-neotetraose (LNnT).

In an additional and/or alternative preferred embodiment, the composition according to this disclosure does not comprise a neutral mammalian milk oligosaccharide. Likewise, it is a preferred embodiment that the method for preventing and/or treating an inflammatory disease or an autoimmune disease according to this disclosure does not comprise the administration of a neutral mammalian milk oligosaccharide.

In an additional and/or alternative preferred embodiment, the composition according to this disclosure does not comprise N-acetyllactosamine. Likewise, it is a preferred embodiment that the method for preventing and/or treating an inflammatory disease or an autoimmune disease according to this disclosure does not comprise the administration of N-acetyllactosamine.

In an additional and/or alternative preferred embodiment, the composition according to this disclosure does not comprise an oligosaccharide containing N-acetyllactosamine, preferably does not comprise a saccharide containing N-acetyllactosamine. Likewise, it is a preferred embodiment that the method for preventing and/or treating an inflammatory disease or an autoimmune disease according to this disclosure does not comprise the administration of an oligosaccharide containing N-acetyllactosamine, preferably does not comprise the administration of a saccharide containing N-acetyllactosamine.

In an additional and/or alternative preferred embodiment, the composition according to this disclosure does not comprise a fructo-oligosaccharide (FOS). Likewise, it is a preferred embodiment that the method for preventing and/or treating an inflammatory disease or an autoimmune disease according to this disclosure does not comprise the administration of a fructo-oligosaccharide (FOS).

In an additional and/or alternative preferred embodiment, the composition according to this disclosure does not comprise a galactose-oligosaccharide (GOS). Likewise, it is a preferred embodiment that the method for preventing and/or treating an inflammatory disease or an autoimmune disease according to this disclosure does not comprise the administration of a galactose-oligosaccharide (GOS).

In an additional and/or alternative preferred embodiment, the composition according to this disclosure does not comprise an isomaltooligosaccharide. Likewise, it is a preferred embodiment that the method for preventing and/or treating an inflammatory disease or an autoimmune disease according to this disclosure does not comprise the administration of isomaltooligosaccharide.

In a more preferred embodiment, the composition according to this disclosure comprises only a sialylated saccharide according to this disclosure (it is referred to in the Section “Saccharide”). Likewise, it is a more preferred embodiment that the method according to this disclosure only comprises the administration of a sialylated saccharide according to this disclosure (it is referred to in the Section “Saccharide”). As elaborated earlier herein, it is most preferred that the method according to this disclosure only comprises the administration of a first sialylated saccharide and a second sialylated saccharide.

In another preferred embodiment, the sialylated saccharide according to this disclosure is present at 0.001-10.000 wt. %, preferably 0.001-7.500 wt. %, more preferably 0.001-5.000 wt. %, even more preferably 0.001-2.500 wt. %, even more preferably 0.001-2.000 wt. %, even more preferably 0.001-1.500 wt. %, most preferably 0.001-1.000 wt. %, of the total weight of the composition.

More preferably, the sialylated saccharide according to this disclosure is present at 0.005-10.000 wt. %, preferably 0.005-7.500 wt. %, more preferably 0.005-5.000 wt. %, even more preferably 0.005-2.500 wt. %, even more preferably 0.005-2.000 wt. %, even more preferably 0.005-1.500 wt. %, most preferably 0.005-1.000 wt. %, of the total weight of the composition.

Even more preferably, the sialylated saccharide according to this disclosure is present at 0.010-10.000 wt. %, preferably 0.010-7.500 wt. %, more preferably 0.010-5.000 wt. %, even more preferably 0.010-2.500 wt. %, even more preferably 0.010-2.000 wt. %, even more preferably 0.010-1.500 wt. %, most preferably 0.010-1.000 wt. %, of the total weight of the composition.

Even more preferably, the sialylated saccharide according to this disclosure is present at 0.100-10.000 wt. %, preferably 0.100-7.500 wt. %, more preferably 0.100-5.000 wt. %, even more preferably 0.100-2.500 wt. %, even more preferably 0.100-2.000 wt. %, even more preferably 0.100-1.500 wt. %, most preferably 0.100-1.000 wt. %, of the total weight of the composition.

Most preferably, the sialylated saccharide according to this disclosure is present at 0.500-10.000 wt. %, preferably 0.500-7.500 wt. %, more preferably 0.500-5.000 wt. %, even more preferably 0.500-2.500 wt. %, even more preferably 0.500-2.000 wt. %, even more preferably 0.500-1.500 wt. %, most preferably 0.500-1.000 wt. %, of the total weight of the composition.

Throughout the disclosure and claims, unless specifically stated otherwise, the term “wt. %” means weight by weight (i.e., w/w). If the composition is, for example, a solid composition, then 1.0 wt. % means 1.0 g sialylated saccharide per 100.0 gram of the solid composition. If the composition is, for example, a liquid composition, then 1.0 wt. % means 1.0 g sialylated saccharide per 100.0 gram of the liquid composition.

In an additional and/or alternative preferred embodiment, the sialylated saccharide according to this disclosure is provided as a powder in the composition according to this disclosure. Preferably, the powder is as defined earlier in the Section “Saccharide.”

As elaborated earlier herein, in some embodiments of this disclosure, two or more sialylated saccharides, preferably two sialylated saccharides (i.e., a first sialylated saccharide and a second sialylated saccharide), are administered to the subject. For the sake of clarity, the expression “preferably two sialylated saccharides” in this context of this disclosure means that no additional sialylated saccharides are administered to the subject. All the embodiments relating to “a sialylated saccharide” of the Section “Composition” apply to “first sialylated saccharide” as described in the disclosure and claims (i.e., according to this disclosure), unless specifically stated otherwise. In other words, each embodiment disclosed in the context of “a sialylated saccharide” is considered to be explicitly disclosed in the context of “a first sialylated saccharide,” unless specifically stated otherwise.

It is a preferred embodiment that the second sialylated saccharide is as described earlier herein in the context of a sialylated saccharide. In other words, each embodiment disclosed in the context of “a sialylated saccharide” is considered to be explicitly disclosed in the context of “a second sialylated saccharide,” unless specifically stated otherwise.

Further, it is a more preferred embodiment that the amount of the first sialylated saccharide in the composition is 0.4 to 50.0, preferably 0.4 to 45.0, more preferably 0.4 to 40.0, even more preferably 0.4 to 35.0, most preferably 0.4 to 30.0, times the amount of the second sialylated saccharide in the composition. In an even more preferred embodiment, the amount of the first sialylated saccharide in the composition is higher than the amount of the second sialylated saccharide in the composition, preferably the amount of the first sialylated saccharide is 1.1 to 50.0, preferably 1.1 to 45.0, more preferably 1.1 to 40.0, even more preferably 1.1 to 35.0, even more preferably 1.1 to 30.0, even more preferably 1.1 to 25.0, even more preferably 1.1 to 20.0, even more preferably 1.1 to 15.0, even more preferably 1.1 to 12.5, even more preferably 1.1 to 10.0, even more preferably 1.5 to 10.0, even more preferably 2.5 to 10, even more preferably 5.0 to 10.0, even more preferably 7.5 to 10.0, most preferably 8.0 to 10.0, times higher than the amount of the second sialylated saccharide in the composition.

In this context, it is an additional and/or alternative more preferred embodiment that the ratio between (i) the amount of the first sialylated saccharide in the composition and (ii) the amount of the second sialylated saccharide in the composition is similar to this ratio as found in the mother's milk of the same species. Throughout the disclosure and claims, unless specifically stated otherwise, a “similar” ratio preferably refers to a ratio that is identical or falls in the range of ratio +/−20% of the ratio, preferably ratio +/−10% of the ratio. For clarity, this means that when the subject is a dog, it is particularly preferred that the ratio between (i) the amount of the first sialylated saccharide being, for example, 3′SL in the composition and (ii) the amount of the second sialylated saccharide being, for example, 6′SL in the composition is similar to the 3′SL/6′SL ratio as generally found in mother's milk of a dog, i.e., 9:1+/−1.8 (in other words 7.2-10.9), preferably 9:1+/−0.9 (in other words 8.2-10.0).

When the subject is a human, then it is preferred that the ratio between (i) the amount of the first sialylated saccharide in the composition and (ii) the amount of the second sialylated saccharide in the composition is 0.4-4.0, preferably 0.4-3.5, more preferably 0.4-3.0, even more preferably 0.4-2.5, even more preferably 0.4-2.0, even more preferably 0.6-2.0, and most preferably 1.0-2.0.

When the subject is a dog or horse, then it is preferred that the ratio between (i) the amount of the first sialylated saccharide in the composition and (ii) the amount of the second sialylated saccharide in the composition is 1.1 to 15.0, preferably 1.1 to 12.5, more preferably 1.1 to 10.0, even more preferably 1.5 to 10.0, even more preferably 2.5 to 10, even more preferably 5.0 to 10.0, even more preferably 7.5 to 10.0, and most preferably 8.0 to 10.0.

When the subject is a bovine, then it is preferred that the ratio between (i) the amount of the first sialylated saccharide in the composition and (ii) the amount of the second sialylated saccharide in the composition is 1.5 to 6.0, preferably 1.5 to 5.0, more preferably 1.5 to 4.0, even more preferably 1.5 to 3.5, even more preferably 2.0 to 3.5, and most preferably 2.5 to 3.5.

When the subject is a pig or a cat, then it is preferred that the ratio between (i) the amount of the first sialylated saccharide in the composition and (ii) the amount of the second sialylated saccharide in the composition is 7.5-40.0, preferably 7.5-35.0, more preferably 10.0-35.0, even more preferably 15.0-35.0, even more preferably 20.0-35.0, and most preferably 25.0-35.0.

When the subject is a goat, then it is preferred that the ratio between (i) the amount of the first sialylated saccharide in the composition and (ii) the amount of the second sialylated saccharide in the composition is 0.25-5.0, preferably 0.25-3.0, more preferably 0.25-2.0, even more preferably 0.25-1.5, and most preferably 0.5-1.5.

Throughout the disclosure and claims, the “amount” is preferably expressed as weight in the context of administration and preferably expressed as w/w in the context of a composition.

Method for Preventing and/or Treating Inflammatory Disease and/or Autoimmune Disease

In a second aspect, this disclosure provides a method for preventing and/or treating an inflammatory disease or an autoimmune disease in a subject. The method comprises administering a sialylated saccharide.

Preferably, this disclosure provides a method for preventing and/or treating an inflammatory disease or an autoimmune disease in a subject. The method comprises administering a composition comprising a sialylated saccharide.

In the context of this disclosure, the “method for preventing and/or treating,” “inflammatory disease,” “autoimmune disease,” “subject,” “composition” and “sialylated saccharide” as described throughout the second aspect of this disclosure and claims are as described in the first aspect of this disclosure. In other words, each embodiment according to the first aspect of this disclosure also applies in the context of the second aspect of this disclosure.

In a second aspect, this disclosure further provides a method for promoting the polarization of peripheral blood mononuclear CD14+ cells, preferably CD14+ monocytes, toward a less inflammatory status in a subject. The method comprises administering a sialylated saccharide.

Preferably, this disclosure provides a method for promoting the polarization of peripheral blood mononuclear CD14+ cells, preferably CD14+ monocytes, toward a less inflammatory status in a subject. The method comprises administering a composition comprising a sialylated saccharide.

In the context of this disclosure, the “subject,” “composition” and “sialylated saccharide” as described throughout the second aspect of this disclosure and claims are as described in the first aspect of this disclosure. In other words, each embodiment according to the first aspect of this disclosure also applies in the context of the second aspect of this disclosure.

### Use

In a third aspect, this disclosure provides the use of a sialylated saccharide for the manufacture of a medicament for preventing and/or treating an inflammatory disease or an autoimmune disease in a subject.

Preferably, this disclosure provides the use of a composition for the manufacture of a medicament for preventing and/or treating an inflammatory disease or an autoimmune disease in a subject, wherein the composition comprises a sialylated saccharide.

In the context of this disclosure, the “method for preventing and/or treating,” “inflammatory disease,” “autoimmune disease,” “subject,” “composition” and “sialylated saccharide” as described throughout the third aspect of this disclosure and claims are as described in the first aspect of this disclosure. In other words, each embodiment according to the first aspect of this disclosure also applies in the context of the third aspect of this disclosure.

In a third aspect, this disclosure further provides the use of a sialylated saccharide for the manufacture of a medicament for promoting the polarization of peripheral blood mononuclear CD14+ cells, preferably CD14+ monocytes, toward a less inflammatory status in a subject.

Preferably, this disclosure provides the use of a composition for the manufacture of a medicament for promoting the polarization of peripheral blood mononuclear CD14+ cells, preferably CD14+ monocytes, toward a less inflammatory status in a subject.

In the context of this disclosure, the “subject,” “composition” and “sialylated saccharide” as described throughout the third aspect of this disclosure and claims are as described in the first aspect of this disclosure. In other words, each embodiment according to the first aspect of this disclosure also applies in the context of the third aspect of this disclosure.

Method for Maintaining and/or Improving Mobility

In a fourth aspect, this disclosure provides a method for maintaining or improving, preferably improving, mobility in a healthy subject. The method comprises administering a sialylated saccharide.

Preferably, this disclosure provides a method for maintaining or improving, preferably improving, mobility in a healthy subject. The method comprises administering a composition comprising a sialylated saccharide. For the sake of clarity, the method according to the fourth aspect of this disclosure is a non-therapeutic method as a healthy subject is ‘treated’ with the sialylated saccharide.

In the context of this disclosure, the “subject,” “composition” and “sialylated saccharide” as described throughout the fourth aspect of this disclosure and claims are as described in the first aspect of this disclosure (it is referred to in the Sections “Subject,” “Composition” and “Saccharide,” respectively, of this disclosure). Furthermore, each embodiment disclosed in the Section “Method for preventing and/or treating” of the first aspect of this disclosure is also disclosed in the context of the method according to the fourth aspect of this disclosure. For example, it is a preferred embodiment of a method according to the fourth invention that the method comprises administering an effective amount of the sialylated saccharide (it is referred to in the Section “Saccharide”) to the healthy subject. For example, it is a more preferred embodiment that the sialylated saccharide is administered at a daily dose of at least 1.0 mg, preferably at least 10.0 mg, more preferably at least 50.0 mg, even more preferably at least 100.0 mg, even more preferably at least 250.0 mg, even more preferably at least 500.0 mg, even more preferably at least 1.0 g, most preferably at least 1.5 g. For example, it is an additional and/or alternative more preferred embodiment that the sialylated saccharide is administered at a daily dose of 0.001-15.0 g, preferably 0.001-12.5 g, more preferably 0.001-10.0 g, even more preferably 0.001-7.5 g, even more preferably 0.001-5.0 g, most preferably 0.001-2.5 g.

For the sake of clarity, the Section “Mode of action” of the first aspect of this disclosure preferably applies in the context of the fourth aspect of this disclosure.

In the context of this disclosure, the term “healthy subject” refers to a subject who has not been diagnosed by a physician as having osteoarthritis, preferably a subject who has not been diagnosed by a physician as having arthritis (preferably as described in the Section “Inflammatory disease/autoimmune disease” of the first aspect of this disclosure).

It is a preferred embodiment that the healthy subject suffers from one or more selected from joint discomfort, joint stiffness and joint pain, more preferably the healthy subject suffers from joint discomfort and/or joint pain.

In an additional and/or preferred embodiment, the subject is a subject as disclosed in the Section “Subject” of the first aspect of this disclosure.

In an additional and/or alternative embodiment, the subject, preferably wherein the subject is a human, does recreational or professional sports. If the subject, preferably wherein the subject is a human, does sports, the sports impacts the joints of the subject, that suffers from one or more selected from joint discomfort, joint stiffness and joint pain, more preferably the healthy subject suffers from joint discomfort and/or joint pain. If the subject, preferably wherein the subject is a human, does sports, the subject recovers faster from the impact of the sport on the joints and bones when a sialylated saccharide according to this disclosure is administered.

In this context of this disclosure, the healthy subject is preferably a human. If the subject is a human, it is a preferred embodiment that the subject is at least 40, preferably at least 45, more preferably at least 50, years old. Preferably, the human does recreational or professional sports. If the human does sports, the sports impact the joints of the human, who suffers from one or more selected from joint discomfort, joint stiffness and joint pain, more preferably the healthy subject suffers from joint discomfort and/or joint pain. If the human does sports, the human recovers faster from the impact of the sport on the joints and bones when a sialylated saccharide according to this disclosure is administered.

In the context of this disclosure, the term “mobility” refers to the ability of a subject to move. Maintenance or improvement in mobility can be measured by various methods known in the art. Suitable methods include single-leg-step-down (SLSD; Schön et al, 2021, Sports Med. Open. 7:24), 6-minute walking test (Macias-Hernandez et al, 2016, Clin. Rheum. 35(8): p. 2087-2092), gait speed test, range of motion test (movement of joint measured using a goniometer), force plate analysis, short physical performance battery and self-reported mobility. Preferably, SLSD, 6-minute walking test, gait speed test or force plate analysis are used to assess mobility. More preferably, SLSD, 6-minute walking test or gait speed test are used to assess mobility. Most preferably, SLSD is used to assess mobility. SLSD has proven to be a reliable tool to assess mobility in healthy subjects, subjects with osteoarthritis and subject with activity-related joint discomfort (without any diagnosed knee joint disease) (Schön et al, supra). While not a prerequisite, it is preferred that the same method is used to follow-up the mobility of a subject and hence assess whether the mobility of the subject is maintained/improved or not.

In the context of this disclosure, the terms “maintain,” “maintains” and “maintaining” refers to a certain parameter, e.g., mobility in the present context, remaining substantially unchanged over a period of time. The period of time is preferably 2 weeks, more preferably 1 month, even more preferably 2 months, even more preferably 3 months, even more preferably 6 months, even more preferably 9 months, even more preferably 12 months. Throughout the disclosure and claims, the period of time is preferably the duration of the method according to this disclosure (it is referred to in the Section “Method for preventing and/or treating”). Substantially unchanged preferably means that the mobility of the subject does not increase or decrease by more than 2%, preferably does not increase or decrease by more than 1%, over the period of time.

In a preferred embodiment, the method according to this disclosure improves mobility in the healthy subject. In a more preferred embodiment, the mobility of a healthy subject increases by at least 2%, preferably at least 3%, more preferably at least 5%, even more preferably at least 10%, even more preferably at least 15%, even more preferably at least 20%, preferably over the period of time. The increase can be relative to the mobility of the subject prior to administration of the sialylated saccharide or can be relative to the mobility of a control healthy subject that did not receive the sialylated saccharide, preferably over the same period of time. Preferably, the increase is relative to the mobility of the subject prior to administration of the sialylated saccharide, preferably over the period of time.

In an additional and/or alternative more preferred embodiment, the mobility of a healthy subject is measured by force plate analysis, 6 minute walking test or gait speed test, preferably force plate analysis or 6 minute walking test, more preferably force plate analysis.

Method for Maintaining and/or Improving Joint Function and/or Muscle Strength

In a fifth aspect, this disclosure provides a method for maintaining or improving, preferably improving, joint function and/or muscle strength, preferably joint function and muscle strength, in a healthy subject. The method comprises administering a sialylated saccharide.

Preferably, this disclosure provides a method for maintaining or improving, preferably improving, joint function and/or muscle strength, preferably joint function and muscle strength in a healthy subject. The method comprises administering a composition comprising a sialylated saccharide. For the sake of clarity, the method according to the fifth aspect of this disclosure is a non-therapeutic method as a healthy subject is ‘treated’ with the sialylated saccharide.

In the context of this disclosure, the “subject,” “composition” and “sialylated saccharide” as described throughout the fifth aspect of this disclosure and claims are as described in the first aspect of this disclosure (it is referred to in the Sections “Subject,” “Composition” and “Saccharide,” respectively, of this disclosure). Furthermore, each embodiment disclosed in the Section “Method for preventing and/or treating” of the first aspect of this disclosure is also disclosed in the context of the method according to the fifth aspect of this disclosure. For example, it is a preferred embodiment of a method according to the fifth invention that the method comprises administering an effective amount of the sialylated saccharide (it is referred to in the Section “Saccharide”) to the healthy subject. For example, it is a more preferred embodiment that the sialylated saccharide is administered at a daily dose of at least 1.0 mg, preferably at least 10.0 mg, more preferably at least 50.0 mg, even more preferably at least 100.0 mg, even more preferably at least 250.0 mg, even more preferably at least 500.0 mg, even more preferably at least 1.0 g, most preferably at least 1.5 g. For example, it is an additional and/or alternative more preferred embodiment that the sialylated saccharide is administered at a daily dose of 0.001-15.0 g, preferably 0.001-12.5 g, more preferably 0.001-10.0 g, even more preferably 0.001-7.5 g, even more preferably 0.001-5.0 g, most preferably 0.001-2.5 g.

For the sake of clarity, the Section “Mode of action” of the first aspect of this disclosure preferably applies in the context of the fifth aspect of this disclosure.

In the context of this disclosure, the term “healthy subject” refers to a subject who has not been diagnosed by a physician as having osteoarthritis, preferably a subject who has not been diagnosed by a physician as having arthritis (preferably as described in the Section “Inflammatory disease/autoimmune disease” of the first aspect of this disclosure).

In the context of maintaining/improving joint function or joint function and muscle strength, it is a preferred embodiment that the healthy subject suffers from one or more selected from joint discomfort, joint stiffness and joint pain, more preferably the healthy subject suffers from joint discomfort and/or joint pain. In an additional and/or preferred embodiment, the subject is a subject as disclosed in the Section “Subject” of the first aspect of this disclosure. In an additional and/or alternative embodiment, the subject, preferably wherein the subject is a human, does recreational or professional sports. If the subject, preferably wherein the subject is a human, does sports, the sports impacts the joints of the subject, that suffers from one or more selected from joint discomfort, joint stiffness and joint pain, more preferably the healthy subject suffers from joint discomfort and/or joint pain. If the subject, preferably wherein the subject is a human, does sports, the subject recovers faster from the impact of the sport on the joints and bones when a sialylated saccharide according to this disclosure is administered.

In this context of this disclosure, the healthy subject is preferably a human. If the subject is a human, it is a preferred embodiment that the subject is at least 40, preferably at least 45, more preferably at least 50, years old. Preferably, the human does recreational or professional sports. If the human does sports, the sports impact the joints of the human, who suffers from one or more selected from joint discomfort, joint stiffness and joint pain, more preferably the healthy subject suffers from joint discomfort and/or joint pain. If the human does sports, the human recovers faster from the impact of the sport on the joints and bones when a sialylated saccharide according to this disclosure is administered.

In the context of maintaining/improving muscle strength, it is a preferred embodiment that the healthy subject is a subject as disclosed in the Section “Subject” of the first aspect of this disclosure. In a more preferred embodiment, the healthy subject is a trained subject, preferably an athlete or an animal in sport. The animal in sport is preferably a racing horse or a racing dog, more preferably a racing horse. A trained subject preferably refers to a subject that performs a physical activity at least once, preferably at least twice, more preferably at least thrice, a week. The physical activity refers to any bodily movement produced by skeletal muscles that requires energy expenditure (i.e., the definition of physical activity as provided by the World Health Organization). Non-limiting examples of physical activity include strength exercises, Tai-Chi, balance exercises, aerobic exercises, walking, dancing, jogging, running, cycling or participating in sports, and flexibility exercises such as yoga, stretching and Pilates. In the context of this disclosure the physical activity most preferably refers to a sport. If the subject performs the physical activity, preferably sport, then the trained muscles recover faster and/or increase in strength when a sialylated saccharide according to this disclosure is administered.

In the context of this disclosure, the term “joint function” preferably refers to the ability of a joint to move in its full range of motion, bear weight and perform work. As is known in the art, individual joints have a predetermined range of motion. This range of motion is commonly measured in degrees. For example, hips, knees, ankles, feet, joints of the feet, joints of the toe, shoulders, elbows, wrists, and hand and finger joints all have different ranges of motion, and there are generally accepted values for a normal range of motion in each of these joints. These values would be known to a skilled person in the art.

Throughout the disclosure and claims, the term “muscle strength” preferably refers to skeletal muscle strength.

Joint function and muscle strength can be measured by various methods known in the art (Hislop et al, 2013, “Daniels and Worthingham's muscle testing: techniques of manual examination and performance testing,” 9^{th} edition). A suitable method for assessing joint function and/or muscle strength is the Manual Muscle Test (MMT), which is a standardized set of assessments that measure muscle strength and function (Fan et al, 2010, Intensive Care Med. 36(6): p. 1038-1043). Another suitable method for assessing joint function and/or muscle strength is a dominant leg strength assessment using an isokinetic dynamometer. This assessment can include measuring isokinetic parameters such as torque max flexor, torque max extensor, total work flexor, total work extensor, and ratios thereof. Further, force plate analysis could be used as well (as illustrated in the Examples below). Preferably, the joint function and/or muscle strength is assessed using MMT or dominant leg strength assessment (isokinetic dynamometer).

Muscle strength can be measured by various methods in the art (Hislop et al, 2013, “Daniels and Worthingham's muscle testing: techniques of manual examination and performance testing,” 9^{th} edition). A commonly applied and preferred method is the Oxford Scale, i.e., Medical Research council Manual Muscle Testing Scale (Naqvi and Sherman, 2022, “Muscle strength grading,” StatPearls Publishing, PMID: 28613779, Bookshelf ID: NBK436008). Another well-suited method is the Manual Muscle Test (MMT), which is a standardized set of assessments that measure muscle strength and function (Fan et al, 2010, Intensive Care Med. 36(6): p. 1038-1043).

While not a prerequisite, it is preferred that the same method is used to follow-up the joint function/muscle strength of a subject and hence assess whether the joint function/muscle strength of the subject is maintained/improved or not.

In the context of this disclosure, the terms “maintain,” “maintains” and “maintaining” refers to a certain parameter, e.g., joint function/muscle strength in the present context, remaining substantially unchanged over a period of time. The period of time is preferably 2 weeks, more preferably 1 month, even more preferably 2 months, even more preferably 3 months, even more preferably 6 months, even more preferably 9 months, even more preferably 12 months. Throughout the disclosure and claims, the period of time is preferably the duration of the method according to this disclosure (it is referred to in the Section “Method for preventing and/or treating”). Substantially unchanged preferably means that the joint function/muscle strength of the subject does not increase or decrease by more than 2%, preferably does not increase or decrease by more than 1%, over the period of time.

In a preferred embodiment, the method according to this disclosure improves joint function in the healthy subject. In a more preferred embodiment, the joint function of a healthy subject increases by at least 2%, preferably at least 3%, more preferably at least 5%, even more preferably at least 10%, even more preferably at least 15%, even more preferably at least 20%, preferably over a period of time. The period of time is preferably 2 weeks, more preferably 1 month, even more preferably 2 months, even more preferably 3 months, even more preferably 6 months, even more preferably 9 months, even more preferably 12 months. Throughout the disclosure and claims, the period of time is preferably the duration of the method according to this disclosure (it is referred to in the Section “Method for preventing and/or treating”). The increase can be relative to the joint function of the subject prior to administration of the sialylated saccharide or can be relative to the joint function of a control healthy subject that did not receive the sialylated saccharide, preferably over the same period of time. Preferably, the increase is relative to the joint function of the subject prior to administration of the sialylated saccharide, preferably over the period of time.

In an additional and/or alternative more preferred embodiment, the joint function of a healthy subject is measured by MMT, dominant leg strength assessment (isokinetic dynamometer) or force plate analysis, more preferably by MMT or dominant leg strength assessment (isokinetic dynamometer).

In an additional and/or alternative preferred embodiment, the method according to this disclosure improves muscle strength (preferably skeletal muscle strength) in the healthy subject. In a more preferred embodiment, the muscle strength of a healthy subject increases by at least 2%, preferably at least 3%, more preferably at least 5%, even more preferably at least 10%, even more preferably at least 15%, even more preferably at least 20%, preferably over a period of time. The period of time is preferably 2 weeks, more preferably 1 month, even more preferably 2 months, even more preferably 3 months, even more preferably 6 months, even more preferably 9 months, even more preferably 12 months. Throughout the disclosure and claims, the period of time is preferably the duration of the method according to this disclosure (it is referred to in the Section “Method for preventing and/or treating”). The increase can be relative to the muscle strength of the subject prior to administration of the sialylated saccharide or can be relative to the muscle strength of a control healthy subject that did not receive the sialylated saccharide, preferably over the same period of time. Preferably, the increase is relative to the muscle strength of the subject prior to administration of the sialylated saccharide, preferably over the period of time.

In an additional and/or alternative more preferred embodiment, the muscle strength of a healthy subject is measured using Medical Research council Manual Muscle Testing Scale or Manual Muscle Test (TST), more preferably using Medical Research council Manual Muscle Testing Scale.

### SPECIFIC EMBODIMENTS

This disclosure preferably relates to the following specific embodiments:
1. A sialylated saccharide for use in a method for preventing and/or treating an inflammatory disease and/or autoimmune disease in a subject.
2. A sialylated saccharide for use according to embodiment 1, wherein the method is for preventing and/or treating an inflammatory disease in a subject, preferably wherein the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, antiphospholipid antibody syndrome, myositis, scleroderma, vasculitis, Sjogren's syndrome, asthma, tuberculosis, chronic periodontitis, chronic sinusitis, chronic active hepatitis, Alzheimer's, Parkinson's disease, nephritis, fibromyalgia, atherosclerosis, eczema, gastritis, sinusitis, seborrheic dermatitis, type-2 diabetes and Wegener's granulomatosis.
3. A sialylated saccharide for use according to embodiment 1, wherein the method is for preventing and/or treating an autoimmune disease in a subject, preferably wherein the autoimmune disease is selected from the list consisting of autoimmune myocarditis, Dressier's syndrome, postpericardiotomy syndrome, subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, Systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune Oophoritis, endometriosis, Sjogren syndrome, autoimmune enteropathy, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, chronic Lyme disease, mixed connective tissue disease, palindromic rheumatism, Parry Romberg syndrome, Parsonage-Turner syndrome, psoriatic arthritis, reactive arthritis, relapsing polychondritis, retroperitoneal fibrosis, rhematic fever, rheumatoid arthritis, sarcoidosis, Schnitzler syndrome, systemic lupus erythematosus, undifferentiated connective tissue disease, dermatomyositis, fibromyalgia, inclusion body myositis, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, autoimmune inner ear disease, Meniere's disease, Behget's disease, eosinophilic granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatica, urticarial vasculitis, vasculitis, and primary immune deficiency.
4. A sialylated saccharide for use according to any one of embodiments 1 to 3, wherein the method is for preventing and/or treating a disease that causes joint inflammation in a subject.
5. A sialylated saccharide for use according to any one of embodiments 1 to 4, wherein the method is for preventing and/or treating arthritis in a subject.
6. A sialylated saccharide for use according to any one of embodiments 1 to 5, wherein the method is for preventing and/or treating arthritis selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis, preferably wherein the arthritis is selected from the list consisting of osteoarthritis, rheumatoid arthritis, systemic lupus erythematosus and juvenile arthritis, more preferably wherein the arthritis is osteoarthritis.
7. A sialylated saccharide for use according to any one of embodiments 1 to 6, wherein the method promotes the polarization of peripheral blood mononuclear CD14+ cells, preferably CD14+ monocytes, toward a less inflammatory status.
8. A sialylated saccharide for use according to any one of embodiments 1 to 7, wherein the method downregulates the tumor necrosis factor alpha (TNF-α) signaling via NF-κB (nuclear factor kappa B) pathway and/or downregulating the IL-6/JAK/STAT3 signaling pathway in peripheral blood mononuclear CD14+ cells, preferably CD14+ monocytes.
9. A sialylated saccharide for use according to any one of embodiments 1 to 8, wherein the subject is a human or an animal, preferably a human or a mammal.
10. A sialylated saccharide for use according to any one of embodiments 1 to 9, wherein the sialylated saccharide comprises a sialic acid, preferably a N-acetylneuraminic acid (Neu5Ac), that is linked to a monosaccharide in an alpha-2,3-, alpha-2,6- or alpha-2,8-linkage, preferably an alpha-2,3- or an alpha-2,6-linkage, more preferably an alpha-2,3-linkage, and wherein the monosaccharide is preferably selected from galactose, N-acetylglucosamine and Neu5Ac, more preferably the monosaccharide is galactose or N-acetylglucosamine, even more preferably the monosaccharide is galactose.
11. A sialylated saccharide for use according to any one of embodiments 1 to 10, wherein the sialylated saccharide is an oligosaccharide, preferably wherein the oligosaccharide consists of 3-9, preferably 3-8, more preferably 3-7, even more preferably 3-6, most preferably 3-5, monosaccharides.
12. A sialylated saccharide for use according to any one of embodiments 1 to 11, wherein the sialylated saccharide is a milk saccharide, preferably a mammalian milk saccharide, more preferably a human milk saccharide.
13. A sialylated saccharide for use according to any one of embodiments 1 to 12, wherein the sialylated saccharide comprises a lactose, a lacto-N-biose (LNB) or N-acetyllactosamine (LacNAc) at its reducing end, preferably the sialylated saccharide comprises lactose or LacNAc at its reducing end, most preferably the sialylated saccharide comprises lactose at its reducing end.
14. A sialylated saccharide for use according to any one of embodiments 1 to 13, wherein the sialylated saccharide is selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x.
15. A sialylated saccharide for use according to any one of embodiments 1 to 14, wherein the sialylated saccharide comprises a sialic acid, preferably Neu5Ac, that is linked to a monosaccharide in an alpha-2,3-linkage.
16. A sialylated saccharide for use according to any one of embodiments 1 to 15, wherein the sialylated saccharide is selected from the list consisting of 3′-sialyllactose (3′SL), 3,6-disialyllactose, 8,3-disialyllactose, 3'S-2′FL, 3'S-3-FL, LST a, LST d, 3′-sialyllacto-N-biose (3′SLNB), sialyl Lewis a, 3′-sialyllactosamine (3′SLacNAc), and sialyl Lewis x.
17. A sialylated saccharide for use according to any one of embodiments 1 to 16, wherein the sialylated saccharide is 3′-sialyllactose (3′SL).
18. A sialylated saccharide for use according to any one of embodiments 1 to 17, wherein the sialylated saccharide has been produced, preferably in vitro and/or ex vivo, by a cell, preferably a single cell, wherein the cell is preferably chosen from the list consisting of a microorganism, a plant cell, an animal cell and a protozoan cell.
19. A sialylated saccharide for use according to any one of embodiments 1 to 18, wherein the sialylated saccharide is linked to a carrier for delivery of the sialylated saccharide, preferably wherein the carrier is a ceramide-based carrier or a polypeptide-based carrier, more preferably wherein the carrier is a ceramide-based carrier.
20. A sialylated saccharide for use according to any one of embodiments 1 to 19, wherein the sialylated saccharide is encapsulated.
21. A sialylated saccharide for use according to any one of embodiments 1 to 20, wherein the method comprises administering an effective amount of the sialylated saccharide.
22. A sialylated saccharide for use according to any one of embodiments 1 to 21, wherein the sialylated saccharide is administered at a daily dose of at least 1.0 mg, preferably at least 10.0 mg, more preferably at least 50.0 mg, even more preferably at least 100.0 mg, even more preferably at least 250.0 mg, even more preferably at least 500.0 mg, even more preferably at least 1.0 g, most preferably at least 1.5 g.
23. A sialylated saccharide for use according to any one of embodiments 1 to 22, wherein the sialylated saccharide is administered at a daily dose of 0.001-15.0 g, preferably 0.001-12.5 g, more preferably 0.001-10.0 g, even more preferably 0.001-7.5 g, even more preferably 0.001-5.0 g, most preferably 0.001-2.5 g.
24. A sialylated saccharide for use according to any one of embodiments 1 to 23, wherein the sialylated saccharide is administered at a daily dose of 0.001-500.0 mg, preferably 0.001-250 mg, more preferably 0.001-200.0 mg, even more preferably 0.001-150.0 mg, even more preferably 0.001-125.0 mg, most preferably 0.001-100.0 mg, per kg bodyweight of the subject.
25. A sialylated saccharide for use according to any one of embodiments 1 to 24, wherein the sialylated saccharide is administered to the subject for at least 2, preferably at least 3, more preferably at least 4, even more preferably at least 6, even more preferably at least 8, even more preferably at least 10, most preferably at least 12, consecutive weeks.
26. A sialylated saccharide for use according to any one of embodiments 1 to 25, wherein the sialylated saccharide is administered to the subject for 2-32, preferably 2-32, more preferably 2-32, even more preferably 2-28, even more preferably 2-24, even more preferably 2-20, even more preferably 2-16, most preferably 2-12, consecutive weeks.
27. A sialylated saccharide for use according to any one of embodiments 1 to 26, wherein the sialylated saccharide is part of a composition.
28. A sialylated saccharide for use according to embodiment 27, wherein the composition is a synthetic composition.
29. A sialylated saccharide for use according to embodiment 27 or 28, wherein the composition is a pharmaceutical composition.
30. A sialylated saccharide for use according to any one of embodiments 27 to 29, wherein the composition is a nutritional composition.
31. A sialylated saccharide for use according to any one of embodiments 27 to 30, wherein the sialylated saccharide is present at 0.001-10.000 wt. %, preferably 0.001-7.500 wt. %, more preferably 0.001-5.000 wt. % even more preferably 0.001-2.500 wt. % even more preferably 0.001-2.000 wt. %, even more preferably 0.001-1.500 wt. % most preferably 0.001-1.000 wt. %, of the total weight of the composition.
32. A composition for use in a method for preventing and/or treating an inflammatory disease and/or autoimmune disease in a subject, wherein the composition comprises a sialylated saccharide.
33. A composition for use according to embodiment 32, wherein the method is for preventing and/or treating an inflammatory disease in a subject, preferably wherein the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, antiphospholipid antibody syndrome, myositis, scleroderma, vasculitis, Sjogren's syndrome, asthma, tuberculosis, chronic periodontitis, chronic sinusitis, chronic active hepatitis, Alzheimer's, Parkinson's disease, nephritis, fibromyalgia, atherosclerosis, eczema, gastritis, sinusitis, seborrheic dermatitis, type-2 diabetes and Wegener's granulomatosis.
34. A composition for use according to embodiment 32, wherein the method is for preventing and/or treating an autoimmune disease in a subject, preferably wherein the autoimmune disease is selected from the list consisting of autoimmune myocarditis, Dressier's syndrome, postpericardiotomy syndrome, subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, Systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune Oophoritis, endometriosis, Sjogren syndrome, autoimmune enteropathy, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, chronic Lyme disease, mixed connective tissue disease, palindromic rheumatism, Parry Romberg syndrome, Parsonage-Turner syndrome, psoriatic arthritis, reactive arthritis, relapsing polychondritis, retroperitoneal fibrosis, rhematic fever, rheumatoid arthritis, sarcoidosis, Schnitzler syndrome, systemic lupus erythematosus, undifferentiated connective tissue disease, dermatomyositis, fibromyalgia, inclusion body myositis, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, autoimmune inner ear disease, Meniere's disease, Behget's disease, eosinophilic granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatica, urticarial vasculitis, vasculitis, and primary immune deficiency.
35. A composition for use according to any one of embodiments 32 to 34, wherein the method is for preventing and/or treating a disease that causes joint inflammation in a subject.
36. A composition for use according to any one of embodiments 32 to 35, wherein the method is for preventing and/or treating arthritis in a subject.
37. A composition for use according to any one of embodiments 32 to 36, wherein the method is for preventing and/or treating arthritis selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis, preferably wherein the arthritis is selected from the list consisting of osteoarthritis, rheumatoid arthritis, systemic lupus erythematosus and juvenile arthritis, more preferably wherein the arthritis is osteoarthritis.
38. A composition for use according to any one of embodiments 32 to 37, wherein the method promotes the polarization of peripheral blood mononuclear CD14+ cells, preferably CD14+ monocytes, toward a less inflammatory status.
39. A composition for use according to any one of embodiments 32 to 38, wherein the method downregulates the tumor necrosis factor alpha (TNF-α) signaling via NF-κB (nuclear factor kappa B) pathway and/or downregulating the IL-6/JAK/STAT3 signaling pathway in peripheral blood mononuclear CD14+ cells, preferably CD14+ monocytes.
40. A composition for use according to any one of embodiments 32 to 39, wherein the subject is a human or an animal, preferably a human or a mammal.
41. A composition for use according to any one of embodiments 32 to 40, wherein the method comprises administering an effective amount of the sialylated saccharide to the subject, preferably wherein the sialylated saccharide is administered as defined in any one of embodiments 22 to 26.
42. A composition for use according to any one of embodiments 32 to 41, wherein the sialylated saccharide is present at 0.001-10.000 wt. %, preferably 0.001-7.500 wt. %, more preferably 0.001-5.000 wt. %, even more preferably 0.001-2.500 wt. %, even more preferably 0.001-2.000 wt. %, even more preferably 0.001-1.500 wt. %, most preferably 0.001-1.000 wt. %, of the total weight of the composition.
43. A composition for use according to any one of embodiments 32 to 42, wherein the sialylated saccharide is as defined in any one of embodiments 10 to 20.
44. A composition for use according to any one of embodiments 32 to 43, wherein the composition further comprises a second sialylated saccharide.
45. A composition for use according to embodiment 44, wherein the second sialylated saccharide comprises a sialic acid, preferably a N-acetylneuraminic acid (Neu5Ac), that is linked to a monosaccharide in an alpha-2,3-, alpha-2,6- or alpha-2,8-linkage, preferably an alpha-2,3- or an alpha-2,6-linkage, more preferably an alpha-2,6-linkage, and wherein the monosaccharide is preferably selected from galactose, N-acetylglucosamine and Neu5Ac, more preferably the monosaccharide is galactose or N-acetylglucosamine, even more preferably the monosaccharide is galactose.
46. A composition for use according to embodiment 44 or 45, wherein the second sialylated saccharide is an oligosaccharide, preferably wherein the oligosaccharide consists of 3-9, preferably 3-8, more preferably 3-7, even more preferably 3-6, most preferably 3-5, monosaccharides.
47. A composition for use according to any one of embodiments 44 to 46, wherein the second sialylated saccharide is a milk saccharide, preferably a mammalian milk saccharide, more preferably a human milk saccharide.
48. A composition for use according to any one of embodiments 44 to 47, wherein the second sialylated saccharide comprises a lactose, a lacto-N-biose (LNB) or N-acetyllactosamine (LacNAc) at its reducing end, preferably the sialylated saccharide comprises lactose or LacNAc at its reducing end, most preferably the sialylated saccharide comprises lactose at its reducing end.
49. A composition for use according to any one of embodiments 44 to 48, wherein the second sialylated saccharide is selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x.
50. A composition for use according to any one of embodiments 44 to 49, wherein the second sialylated saccharide comprises a sialic acid, preferably Neu5Ac, that is linked to a monosaccharide in an alpha-2,6-linkage.
51. A composition for use according to any one of embodiments 44 to 50, wherein the second sialylated saccharide is selected from the list consisting of 6′-sialyllactose (6′SL), 3,6-disialyllactose, 6,6-disialyllactose, 6'S-2′FL, 6'S-3-FL, LST b, LST c, 6′-sialyllacto-N-biose (6′SLNB) and 6′-sialyllactosamine (6′SLacNAc).
52. A composition for use according to any one of embodiments 44 to 51, wherein the second sialylated saccharide is 6′-sialyllactose (6′SL).
53. A composition for use according to any one of embodiments 44 to 52, wherein the method comprises administering an effective amount of the second sialylated saccharide to the subject, preferably wherein the second sialylated saccharide is administered as defined in any one of embodiments 22 to 26.
54. A composition for use according to any one of embodiments 44 to 53, wherein the second sialylated saccharide is present at 0.001-10.000 wt. %, preferably 0.001-7.500 wt. %, more preferably 0.001-5.000 wt. % even more preferably 0.001-2.500 wt. %, even more preferably 0.001-2.000 wt. %, even more preferably 0.001-1.500 wt. %, most preferably 0.001-1.000 wt. %, of the total weight of the composition.
55. A composition for use according to any one of embodiments 44 to 54, wherein the amount of the first sialylated saccharide in the composition is 0.4 to 50.0, preferably 0.4 to 45.0, more preferably 0.4 to 40.0, even more preferably 0.4 to 35.0, most preferably 0.4 to 30.0, times the amount of the second sialylated saccharide in the composition.
56. A composition for use according to any one of embodiments 44 to 55, wherein the second sialylated saccharide has been produced, preferably in vitro and/or ex vivo, by a cell, preferably a single cell, wherein the cell is preferably chosen from the list consisting of a microorganism, a plant cell, an animal cell and a protozoan cell.
57. A composition for use according to any one of embodiments 44 to 56, wherein the second sialylated saccharide is linked to a carrier for delivery of the second sialylated saccharide, preferably wherein the carrier is a ceramide-based carrier or a polypeptide-based carrier, more preferably wherein the carrier is a ceramide-based carrier.
58. A composition for use according to any one of embodiments 44 to 57, wherein the second sialylated saccharide is encapsulated.
59. A composition for use according to any one of embodiments 32 to 58, wherein the composition is a synthetic composition.
60. A composition for use according to any one of embodiments 32 to 59, wherein the composition is a pharmaceutical composition.
61. A composition for use according to any one of embodiments 32 to 60, wherein the composition is a nutritional composition.
62. A method for preventing and/or treating an inflammatory disease and/or autoimmune disease in a subject, wherein the method comprises administering a sialylated saccharide.
63. A method according to embodiment 62, wherein the method is for preventing and/or treating an inflammatory disease in a subject, preferably wherein the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, antiphospholipid antibody syndrome, myositis, scleroderma, vasculitis, Sjogren's syndrome, asthma, tuberculosis, chronic periodontitis, chronic sinusitis, chronic active hepatitis, Alzheimer's, Parkinson's disease, nephritis, fibromyalgia, atherosclerosis, eczema, gastritis, sinusitis, seborrheic dermatitis, type-2 diabetes and Wegener's granulomatosis.
64. A method according to embodiment 62, wherein the method is for preventing and/or treating an autoimmune disease in a subject, preferably wherein the autoimmune disease is selected from the list consisting of autoimmune myocarditis, Dressier's syndrome, postpericardiotomy syndrome, subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, Systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune Oophoritis, endometriosis, Sjogren syndrome, autoimmune enteropathy, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, chronic Lyme disease, mixed connective tissue disease, palindromic rheumatism, Parry Romberg syndrome, Parsonage-Turner syndrome, psoriatic arthritis, reactive arthritis, relapsing polychondritis, retroperitoneal fibrosis, rhematic fever, rheumatoid arthritis, sarcoidosis, Schnitzler syndrome, systemic lupus erythematosus, undifferentiated connective tissue disease, dermatomyositis, fibromyalgia, inclusion body myositis, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, autoimmune inner ear disease, Meniere's disease, Behget's disease, eosinophilic granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatica, urticarial vasculitis, vasculitis, and primary immune deficiency.
65. A method according to any one of embodiments 62 to 64, wherein the method is for preventing and/or treating a disease that causes joint inflammation in a subject.
66. A method according to any one of embodiments 62 to 65, wherein the method is for preventing and/or treating arthritis in a subject.
67. A method according to any one of embodiments 62 to 66, wherein the method is for preventing and/or treating arthritis selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis, preferably wherein the arthritis is selected from the list consisting of osteoarthritis, rheumatoid arthritis, systemic lupus erythematosus and juvenile arthritis, more preferably wherein the arthritis is osteoarthritis.
68. A method for maintaining or improving mobility in a healthy subject, wherein the method comprises administering a sialylated saccharide.
69. A method for maintaining or improving joint function and/or muscle strength in a healthy subject, wherein the method comprises administering a sialylated saccharide.
70. A method for preventing and/or treating an inflammatory disease and/or autoimmune disease in a subject, wherein the method comprises administering a composition comprising a sialylated saccharide.
71. A method according to embodiment 70, wherein the method is for preventing and/or treating an inflammatory disease in a subject, preferably wherein the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, antiphospholipid antibody syndrome, myositis, scleroderma, vasculitis, Sjogren's syndrome, asthma, tuberculosis, chronic periodontitis, chronic sinusitis, chronic active hepatitis, Alzheimer's, Parkinson's disease, nephritis, fibromyalgia, atherosclerosis, eczema, gastritis, sinusitis, seborrheic dermatitis, type-2 diabetes and Wegener's granulomatosis.
72. A method according to embodiment 70, wherein the method is for preventing and/or treating an autoimmune disease in a subject, preferably wherein the autoimmune disease is selected from the list consisting of autoimmune myocarditis, Dressier's syndrome, postpericardiotomy syndrome, subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, Systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune Oophoritis, endometriosis, Sjogren syndrome, autoimmune enteropathy, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, chronic Lyme disease, mixed connective tissue disease, palindromic rheumatism, Parry Romberg syndrome, Parsonage-Turner syndrome, psoriatic arthritis, reactive arthritis, relapsing polychondritis, retroperitoneal fibrosis, rhematic fever, rheumatoid arthritis, sarcoidosis, Schnitzler syndrome, systemic lupus erythematosus, undifferentiated connective tissue disease, dermatomyositis, fibromyalgia, inclusion body myositis, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, autoimmune inner ear disease, Meniere's disease, Behget's disease, eosinophilic granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatica, urticarial vasculitis, vasculitis, and primary immune deficiency.
73. A method according to any one of embodiments 70 to 72, wherein the method is for preventing and/or treating a disease that causes joint inflammation in a subject.
74. A method according to any one of embodiments 70 to 73, wherein the method is for preventing and/or treating arthritis in a subject.
75. A method according to any one of embodiments 70 to 74, wherein the method is for preventing and/or treating arthritis selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis, preferably wherein the arthritis is selected from the list consisting of osteoarthritis, rheumatoid arthritis, systemic lupus erythematosus and juvenile arthritis, more preferably wherein the arthritis is osteoarthritis.
76. A method according to any one of embodiments 62 to 75, wherein the method promotes the polarization of peripheral blood mononuclear CD14+ cells, preferably CD14+ monocytes, toward a less inflammatory status.
77. A method according to any one of embodiments 62 to 76, wherein the method downregulates the tumor necrosis factor alpha (TNF-α) signaling via NF-κB (nuclear factor kappa B) pathway and/or downregulating the IL-6/JAK/STAT3 signaling pathway in peripheral mononuclear CD14+ blood cells, preferably CD14+ monocytes.
78. A method according to any one of embodiments 62 to 77, wherein the subject is a human or an animal, preferably a human or a mammal.
79. A method according to any one of embodiments 62 to 78, wherein the sialylated saccharide is as defined in any one of embodiments 10 to 20 and 27.
80. A method according to any one of embodiments 62 to 79, wherein the method comprises administering an effective amount of the sialylated saccharide to the subject, preferably wherein the sialylated saccharide is administered as defined in any one of embodiments 22 to 26.
81. A method according to any one of embodiments 62 to 80, wherein the method comprises administering a second sialylated saccharide, preferably administering an effective amount of the second sialylated saccharide, more preferably wherein the second sialylated saccharide is administered as defined in any one of embodiments 22 to 26.
82. A method according to embodiment 81, wherein the amount of the first sialylated saccharide administered to the subject is 0.4 to 50.0, preferably 0.4 to 45.0, more preferably 0.4 to 40.0, even more preferably 0.4 to 35.0, most preferably 0.4 to 30.0, times the amount of the second sialylated saccharide administered to the subject.
83. A method according to embodiment 81 or 82, wherein the second sialylated saccharide is as defined in any one of embodiments 45 to 52, 56, 57 and 58.
84. A method according to any one of embodiments 70 to 83, wherein the composition is a synthetic composition.
85. A method according to any one of embodiments 70 to 84, wherein the composition is a pharmaceutical composition.
86. A method according to any one of embodiments 70 to 85, wherein the composition is a nutritional composition.
87. A method according to any one of embodiments 70 to 86, wherein the sialylated saccharide is present at 0.001-10.000 wt. %, preferably 0.001-7.500 wt. %, more preferably 0.001-5.000 wt. %, even more preferably 0.001-2.500 wt. %, even more preferably 0.001-2.000 wt. %, even more preferably 0.001-1.500 wt. %, most preferably 0.001-1.000 wt. %, of the total weight of the composition.
88. A method according to any one of embodiments 70 to 87, wherein the second sialylated saccharide is present at 0.001-10.000 wt. %, preferably 0.001-7.500 wt. %, more preferably 0.001-5.000 wt. %, even more preferably 0.001-2.500 wt. %, even more preferably 0.001-2.000 wt. %, even more preferably 0.001-1.500 wt. %, most preferably 0.001-1.000 wt. %, of the total weight of the composition.
89. A method according to any one of embodiments 68 to 88, wherein the amount of the first sialylated saccharide in the composition is 0.4 to 50.0, preferably 0.4 to 45.0, more preferably 0.4 to 40.0, even more preferably 0.4 to 35.0, most preferably 0.4 to 30.0, times the amount of the second sialylated saccharide in the composition.
90. Use of a sialylated saccharide for the manufacture of a medicament for preventing and/or treating an inflammatory disease and/or autoimmune disease in a subject.
91. Use according to embodiment 90, wherein the medicament is for preventing and/or treating an inflammatory disease in a subject, preferably wherein the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, antiphospholipid antibody syndrome, myositis, scleroderma, vasculitis, Sjogren's syndrome, asthma, tuberculosis, chronic periodontitis, chronic sinusitis, chronic active hepatitis, Alzheimer's, Parkinson's disease, nephritis, fibromyalgia, atherosclerosis, eczema, gastritis, sinusitis, seborrheic dermatitis, type-2 diabetes and Wegener's granulomatosis.
92. Use according to embodiment 90, wherein the medicament is for preventing and/or treating an autoimmune disease in a subject, preferably wherein the autoimmune disease is selected from the list consisting of autoimmune myocarditis, Dressier's syndrome, postpericardiotomy syndrome, subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, Systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune Oophoritis, endometriosis, Sjogren syndrome, autoimmune enteropathy, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, chronic Lyme disease, mixed connective tissue disease, palindromic rheumatism, Parry Romberg syndrome, Parsonage-Turner syndrome, psoriatic arthritis, reactive arthritis, relapsing polychondritis, retroperitoneal fibrosis, rhematic fever, rheumatoid arthritis, sarcoidosis, Schnitzler syndrome, systemic lupus erythematosus, undifferentiated connective tissue disease, dermatomyositis, fibromyalgia, inclusion body myositis, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, autoimmune inner ear disease, Meniere's disease, Behget's disease, eosinophilic granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatica, urticarial vasculitis, vasculitis, and primary immune deficiency.
93. Use according to any one of embodiments 90 to 92, wherein the medicament is for preventing and/or treating a disease that causes joint inflammation in a subject.
94. Use according to any one of embodiments 90 to 93, wherein the medicament is for preventing and/or treating arthritis in a subject.
95. Use according to any one of embodiments 90 to 94, wherein the medicament is for preventing and/or treating arthritis selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis, preferably wherein the arthritis is selected from the list consisting of osteoarthritis, rheumatoid arthritis, systemic lupus erythematosus and juvenile arthritis, more preferably wherein the arthritis is osteoarthritis.
96. Use of a composition for the manufacture of a medicament for preventing and/or treating an inflammatory disease and/or autoimmune disease in a subject, wherein the composition comprises a sialylated saccharide.
97. Use according to embodiment 96, wherein the medicament is for preventing and/or treating an inflammatory disease in a subject, preferably wherein the inflammatory disease is selected from the list consisting of osteoarthritis, rheumatoid arthritis, psoriatic arthritis, juvenile idiopathic arthritis, ankylosing spondylitis, systemic lupus erythematosus, gout, enteropathic arthritis, antiphospholipid antibody syndrome, myositis, scleroderma, vasculitis, Sjogren's syndrome, asthma, tuberculosis, chronic periodontitis, chronic sinusitis, chronic active hepatitis, Alzheimer's, Parkinson's disease, nephritis, fibromyalgia, atherosclerosis, eczema, gastritis, sinusitis, seborrheic dermatitis, type-2 diabetes and Wegener's granulomatosis.
98. Use according to embodiment 96, wherein the medicament is for preventing and/or treating an autoimmune disease in a subject, preferably wherein the autoimmune disease is selected from the list consisting of autoimmune myocarditis, Dressier's syndrome, postpericardiotomy syndrome, subacute bacterial endocarditis, anti-glomerular basement membrane nephritis, interstitial cystitis, lupus nephritis, autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease, morphea, pemphigus vulgaris, pityriasis lichenoides et varioliformis acuta, Mucha-Habermann disease, psoriasis, Systemic scleroderma, vitiligo, Addison's disease, autoimmune polyendocrine syndrome type 1, autoimmune polyendocrine syndrome type 2, autoimmune polyendocrine syndrome type 3, autoimmune pancreatitis, diabetes mellitus type 1, autoimmune thyroiditis, Ord's thyroiditis, Graves' disease, autoimmune Oophoritis, endometriosis, Sjogren syndrome, autoimmune enteropathy, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, chronic Lyme disease, mixed connective tissue disease, palindromic rheumatism, Parry Romberg syndrome, Parsonage-Turner syndrome, psoriatic arthritis, reactive arthritis, relapsing polychondritis, retroperitoneal fibrosis, rhematic fever, rheumatoid arthritis, sarcoidosis, Schnitzler syndrome, systemic lupus erythematosus, undifferentiated connective tissue disease, dermatomyositis, fibromyalgia, inclusion body myositis, myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, autoimmune inner ear disease, Meniere's disease, Behget's disease, eosinophilic granulomatosis with polyangiitis, IgA vasculitis, Kawasaki's disease, leukocytoclastic vasculitis, lupus vasculitis, rheumatoid vasculitis, microscopic polyangiitis, polyarteritis nodosa, polymyalgia rheumatica, urticarial vasculitis, vasculitis, and primary immune deficiency.
99. Use according to any one of embodiments 96 to 98, wherein the medicament is for preventing and/or treating a disease that causes joint inflammation in a subject.
100. Use according to any one of embodiments 96 to 99, wherein the medicament is for preventing and/or treating arthritis in a subject.
101. Use according to any one of embodiments 96 to 100, wherein the medicament is for preventing and/or treating arthritis selected from the list consisting of osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia and enthesitis-related arthritis, preferably wherein the arthritis is selected from the list consisting of osteoarthritis, rheumatoid arthritis, systemic lupus erythematosus and juvenile arthritis, more preferably wherein the arthritis is osteoarthritis.
102. Use according to any one of embodiments 90 to 101, wherein the method promotes the polarization of peripheral blood mononuclear CD14+ cells, preferably CD14+ monocytes, toward a less inflammatory status.
103. Use according to any one of embodiments 90 to 102, wherein the method downregulates the tumor necrosis factor alpha (TNF-α) signaling via NF-κB (nuclear factor kappa B) pathway and/or downregulating the IL-6/JAK/STAT3 signaling pathway in peripheral blood mononuclear CD14+ cells, preferably CD14+ monocytes.
104. Use according to any one of embodiments 90 to 103, wherein the subject is a human or an animal, preferably a human or a mammal.
105. Use according to any one of embodiments 90 to 104, wherein the sialylated saccharide is as defined in any one of embodiments 10 to 20 and 27.
106. Use according to any one of embodiments 90 to 105, wherein the method comprises administering an effective amount of the sialylated saccharide to the subject, preferably wherein the sialylated saccharide is administered as defined in any one of embodiments 22 to 26.
107. Use according to any one of embodiments 90 to 106, wherein the method comprises administering a second sialylated saccharide, preferably administering an effective amount of the second sialylated saccharide, more preferably wherein the second sialylated saccharide is administered as defined in any one of embodiments 22 to 26.
108. Use according to embodiment 107, wherein the amount of the first sialylated saccharide administered to the subject is 0.4 to 50.0, preferably 0.4 to 45.0, more preferably 0.4 to 40.0, even more preferably 0.4 to 35.0, most preferably 0.4 to 30.0, times the amount of the second sialylated saccharide administered to the subject.
109. Use according to embodiment 107 or 108, wherein the second sialylated saccharide is as defined in any one of embodiments 45 to 52, 56, 57 and 58.
110. Use according to any one of embodiments 96 to 109, wherein the composition is a synthetic composition.
111. Use according to any one of embodiments 96 to 110, wherein the composition is a pharmaceutical composition.
112. Use according to any one of embodiments 96 to 111, wherein the composition is a nutritional composition.
113. Use according to any one of embodiments 96 to 112, wherein the sialylated saccharide is present at 0.001-10.000 wt. %, preferably 0.001-7.500 wt. %, more preferably 0.001-5.000 wt. %, even more preferably 0.001-2.500 wt. %, even more preferably 0.001-2.000 wt. %, even more preferably 0.001-1.500 wt. %, most preferably 0.001-1.000 wt. %, of the total weight of the composition.
114. Use according to any one of embodiments 96 to 113, wherein the second sialylated saccharide is present at 0.001-10.000 wt. %, preferably 0.001-7.500 wt. %, more preferably 0.001-5.000 wt. %, even more preferably 0.001-2.500 wt. % even more preferably 0.001-2.000 wt. %, even more preferably 0.001-1.500 wt. %, most preferably 0.001-1.000 wt. %, of the total weight of the composition.
115. Use according to any one of embodiments 96 to 114, wherein the amount of the first sialylated saccharide is higher than the amount of the second sialylated saccharide, preferably the amount of the first sialylated saccharide in the composition is 0.4 to 50.0, preferably 0.4 to 45.0, more preferably 0.4 to 40.0, even more preferably 0.4 to 35.0, most preferably 0.4 to 30.0, times the amount of the second sialylated saccharide in the composition.

### Definitions

The words used in this disclosure to describe the invention and its various embodiments are to be understood not only in the sense of their commonly defined meanings, but to include by special definition in this disclosure structure, material or acts beyond the scope of the commonly defined meanings. Thus, if an element can be understood in the context of this disclosure as including more than one meaning, then its use in a claim must be understood as being generic to all possible meanings supported by the disclosure and by the word itself.

The various aspects and embodiments of the invention disclosed herein are to be understood not only in the order and context specifically described in this disclosure, but to include any order and any combination thereof. Each embodiment as identified herein may be combined together unless otherwise indicated. All publications, patents and patent applications cited in this disclosure are herein incorporated by reference in their entirety to the same extent as if each individual publication, patent or patent application was specifically and individually indicated to be incorporated by reference in its entirety. Unless specifically stated otherwise, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the invention herein and within the scope of this invention, which is limited only by the claims, construed in accordance with the patent law, including the doctrine of equivalents. In the claims that follow, reference characters used to designate claim steps are provided for convenience of description only and are not intended to imply any particular order for performing the steps (unless specifically stated otherwise).

In this disclosure and in its claims, the verbs “to comprise,” “to have” and “to contain,” and their conjugations are used in their non-limiting sense to mean that items following the word are included, but items not specifically mentioned are not excluded. The verb “to consist essentially of” means that e.g., a composition as defined herein may comprise additional component(s) than the ones specifically identified, the additional component(s) not altering the unique characteristic of the invention. Throughout the disclosure and claims, unless specifically stated otherwise, the verbs “to comprise,” “to have” and “to contain,” and their conjugations, may be preferably replaced by “to consist” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one.” The word “about” or “approximately” or “around” when used in association with a numerical value (e.g., about 10) preferably means that the value may be the given value (of 10) more or less 10%, preferably 5%, more preferably 1% of the value.

Throughout the description and claims, unless specifically stated otherwise, the expression “from x to y,” wherein x and y represent numerical values, refers to a range of numerical values wherein both x and y are included and wherein x represents the lowest value and y represents the highest value. Hence, both x and y are included in the range.

The terms “LNT II,” “LNT-II,” “LN3,” “lacto-N-triose II,” “lacto-N-triose II,” “lacto-N-triose,” “lacto-N-triose” and “GlcNAc-β1,3-Gal-β1,4-Glc” are used interchangeably.

The terms “LNT,” “lacto-N-tetraose,” “lacto-N-tetraose” and “Gal-β1,3-GlcNAc-β1,3-Gal-β1,4Glc” are used interchangeably.

The terms “LNnT,” “lacto-N-neotetraose,” “lacto-N-neotetraose,” “neo-LNT” and “Galβ1-4GlcNAcβ1-3Galβ1-4Glc” are used interchangeably.

The terms “2′ fucosyllactose,” “2′-fucosyllactose,” “alpha-1,2-fucosyllactose,” “alpha 1,2 fucosyllactose,” “α-1,2-fucosyllactose,” “α 1,2 fucosyllactose,” “Fuc-α1,2-Gal-β1,4-Glc,” 2FL” and “2′FL” are used interchangeably.

The terms “3-fucosyllactose,” “alpha-1,3-fucosyllactose,” “alpha 1,3 fucosyllactose,” “α-1,3-fucosyllactose,” “α 1,3 fucosyllactose,” “Gal-β1,4-(Fuc-α1,3-)Glc,” 3FL” and “3-FL” are used interchangeably.

The terms “3′ sialyllactose,” “3′-sialyllactose,” “alpha-2,3-sialyllactose,” “alpha 2,3 sialyllactose,” “α-2,3-sialyllactose,” “α 2,3 sialyllactose,” “3SL,” “Sia-α2,3-Gal-β1,4-Glc” and “3′SL” are used interchangeably.

The terms “6′ sialyllactose,” “6′-sialyllactose,” “alpha-2,6-sialyllactose,” “alpha 2,6 sialyllactose,” “α-2,6-sialyllactose,” “α 2,6 sialyllactose,” “6SL,” “Sia-α2,6-Gal-β1,4-Glc” and “6′SL” are used interchangeably.

The terms “3,6-disialyllactose” and “Neu5Ac-α2,3-Neu5Ac-α2,6-Gal-β1,4-GlC” are used interchangeably.

The terms “6,6′-disialyllactose” and “Neu5Ac-α2,6-Neu5Ac-α2,6-Gal-β1,4-Glc” are used interchangeably.

The terms “8,3-disialyllactose” and “Neu5Ac-α2,8-Neu5Ac-α2,3-Gal-β1,4-GlC” are used interchangeably.

The terms “3'S-2′FL,” “3′-sialyl-2′-fucosyllactose” and “Neu5Ac-α2,3-[Fuc-α1,2-]Gal-β1,4-GlC” are used interchangeably.

The terms “6'S-2′FL,” “6′-sialyl-2′-fucosyllactose” and “Neu5Ac-α2,6-[Fuc-α1,2-]Gal-β1,4-GlC” are used interchangeably.

The terms “3'S-3-FL,” “3′-sialyl-3-fucosyllactose” and “Neu5Ac-α2,3-Gal-β1,4-[Fuc-α1,3]GlC” are used interchangeably.

The terms “6'S-3-FL,” “6′-sialyl-3-fucosyllactose” and “Neu5Ac-α2,6-Gal-β1,4-[Fuc-α1,3]GlC” are used interchangeably.

The terms “LSTa,” “LS-Tetrasaccharide a,” “Sialyl-lacto-N-tetraose a,” “sialyllacto-N-tetraose a” and “Neu5Ac-α2,3-Gal-b1,3-GlcNAc-b1,3-Gal-b1,4-Glc” are used interchangeably.

The terms “LSTb,” “LS-Tetrasaccharide b,” “Sialyl-lacto-N-tetraose b,” “sialyllacto-N-tetraose b” and “Gal-b1,3-(Neu5Ac-α2,6)-GlcNAc-b1,3-Gal-b1,4-Glc” are used interchangeably.

The terms “LSTc,” “LS-Tetrasaccharide c,” “Sialyl-lacto-N-tetraose c,” “sialyllacto-N-tetraose c,” “sialyllacto-N-neotetraose c” and “Neu5Ac-α2,6-Gal-b1,4-GlcNAc-b1,3-Gal-b1,4-Glc” are used interchangeably.

The terms “LSTd,” “LS-Tetrasaccharide d,” “Sialyl-lacto-N-tetraose d,” “sialyllacto-N-tetraose d,” “sialyllacto-N-neotetraose d” and “Neu5Ac-α2,3-Gal-b1,4-GlcNAc-b1,3-Gal-b1,4-Glc” are used interchangeably.

The terms “3′-sialyllacto-N-biose,” “3′SLNB” and “Neu5Ac-α2,3-Gal-b1,3-GlcNAc” are used interchangeably.

The terms “6′-sialyllacto-N-biose,” “6′SLNB” and “Neu5Ac-α2,6-Gal-b1,3-GlcNAc” are used interchangeably.

The terms “monofucosylmonosialyllacto-N-octaose,” “sialyl Lewis a,” “sialyl Lea,” “5-acetylneuraminyl-(2-3)-galactosyl-(1-3)-(fucopyranosyl-(1-4))-N-acetylglucosamine” and “Neu5Ac-α2,3-Gal-β1,3-[Fuc-α1,4]-GlcNAc” are used interchangeably.

The terms “3′-sialyllactosamine,” “3′SLacNAc” and “Neu5Ac-α2,3-Gal-b1,4-GlcNAc” are used interchangeably.

The terms “6′-sialyllactosamine,” “6′SLacNAc” and “Neu5Ac-α2,6-Gal-b1,4-GlcNAc” are used interchangeably.

The terms “sialyl Lewis x,” “sialyl Lex,” “5-acetylneuraminyl-(2-3)-galactosyl-(1-4)-(fucopyranosyl-(1-3))-N-acetylglucosamine” and “Neu5Ac-α2,3-Gal-β1,4-[Fuc-α1,3-]GlcNAc” are used interchangeably.

The term “cultivation” refers to the culture medium wherein the cell is cultivated or fermented, the cell itself, and the saccharide(s) that is/are produced by the cell in whole broth, i.e., inside (intracellularly) as well as outside (extracellularly) of the cell.

The term “clarifying” refers to the act of treating an aqueous solution to remove suspended particulates and contaminants from the production process, like e.g., cells, cell components, insoluble metabolites and debris, that could interfere with the eventual purification of the oligosaccharide(s) of interest. Such treatment can be carried out in a conventional manner by centrifugation, flocculation, flocculation with optional ultrasonic treatment, gravity filtration, microfiltration, foam separation or vacuum filtration (e.g., through a ceramic filter that can include a Celite™ filter aid).

### EXAMPLES

The invention will be described in more detail in the examples. The following examples will serve as further illustration and clarification of this disclosure and are not intended to be limiting in any way.

### Example 1: 3′SL and 6′SL

3′sialyllactose (3′SL) and 6′sialyllactose (6′SL) were recombinantly produced in E. coli and purified as described in WO 2022/034079 (Examples 11, 13 and 14; the E. coli strains are described in Example 3 of WO 2018/122225 for 6′SL and Example 7 of WO 2018/122225 for 3′SL wherein an alpha-2,3-sialyltransferase from Pasteurella multocida, i.e., amino acids 1-268 of Uniprot ID Q9CLP3 sequence v1, was used) and subsequently spray dried as described in Example 21 of WO 2022/034079 to obtain 3′SL powder (purity 98.4%) and 6′SL powder (purity 98.6%).

### Example 2: Trial in Dogs

### Animals

Twelve laboratory dogs, 4 Beagleton and 8 Beagle, participated in a double-blinded trial approved by the ethical committee (Project number AVD1080020184847 under Work protocol number 4847-1-13 for companion animals with mild level of discomfort). Eight dogs were classified as having osteoarthritis (OA), while the other four dogs were classified as healthy (both groups had similar age). The classification was based on force plate analyses as elaborated further and determined by specialists at the Utrecht University Faculty of Veterinary Medicine. Dogs from the healthy (i.e., control or non-OA dogs) and OA groups were randomly assigned to be fed with 3′sialyllactose and 6′sialyllactose (i.e., sialyllactose or SL for ease of reference) or without sialyllactose (placebo) to arrive at four groups: healthy-placebo (2 dogs); healthy-SL (2 dogs); OA-placebo (4 dogs) and OA-SL (4 dogs).

The dogs are placed in group housing. The housing exists out of a kennel, inside and outside. Moreover, grass fields are present outside, the dogs are placed here every day for some hours, dependent on the weather. The dogs are individual or with two placed in a kennel, dependent on the behavior to each other. Enrichment is present like toys, balls and blankets. Each animal is checked every day by the animal takers for abnormalities. Each week the dogs are checked on behavior, posture, gate/mobility, food intake/weight loss, grooming/auto mutilation and other striking clinical symptoms. Findings of these welfare checks are noted in the Welfare Logbook. All dogs got deworming once a month (Drontal Large Dog Tasty from Bayer B.V.).

### Diet

The placebo diet consisted out of Hill's Science plan Medium Adult containing corn, wheat, lamb meal, soy flour, animal fat, corn gluten meal, brewer's rice, protein hydrolysate, vegetable oil, linseed and minerals. The diet with sialyllactose was made by mixing the placebo diet with sialyllactose (6 g/kg diet), wherein the sialyllactose consists of 3′sialyllactose and 6′sialyllactose in α 9:1 ratio (i.e., 5.4 g 3′SL/kg diet and 0.6 g 6′SL/kg diet).

Dogs were fed once a day between 7 and 8 μm, water was given without restrictions. Dogs treated with sialyllactose received SL at the dose of 100 mg per kg bodyweight per day, i.e., 90 mg 3′sialyllactose and 10 mg 6′sialyllactose per kg bodyweight per day. Depending on the body weight, dogs were fed with the appropriate portion (250 g food per 15 kg bodyweight).

### Force Plate Analysis

A valid and reliable method to test differences in chronic pain in dogs with OA is force plate analysis (Moreau et al., 2003, Veterinary Record 152(11): p. 323-329; Mueller et al., 2007, Veterinaryrecord 160(22): p. 762-765; Brown et al., 2013, J. Veterinary Internal Medicine 27(1): p. 22-30). Hence, with force plate analysis the effect of sialyllactose in dogs with OA can be determined. Force plate analysis was conducted at start (T1), after two weeks (T2), 2 months (T3) and 4 months (T4). Each analysis was based on at least ten useful measurements on each side. Hence, each dog had at least twenty measurements each force plate session.

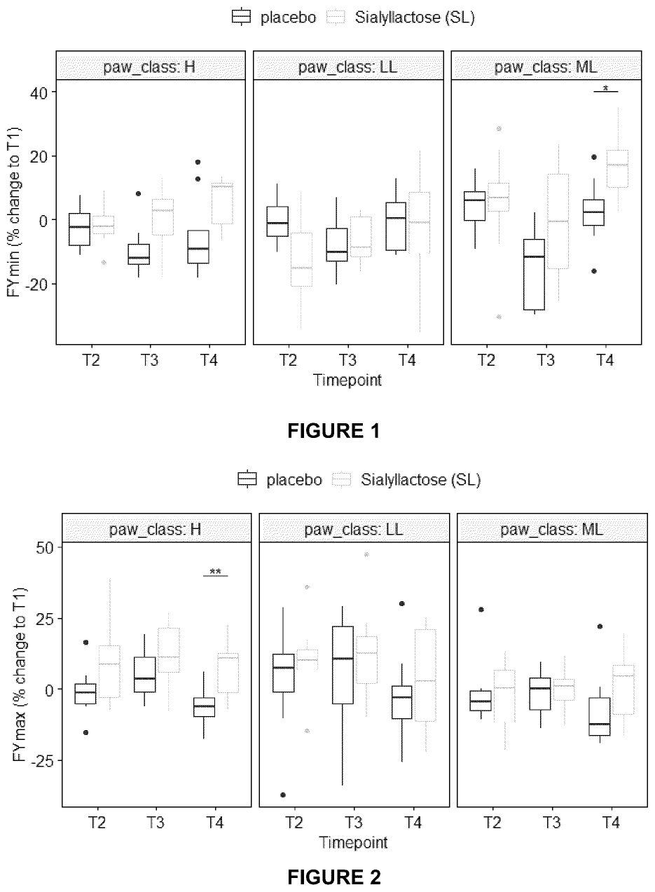

The force plate measurements went as follows. Each dog was taken separately from the kennel to the force plate. At first the bodyweight of the dog was determined by the DIWAC VS150 electronic scale, the weight was filled into the computer system. The type of force plate being used was a Kistler type 9261, a quartz piezoelectric force plate. Furthermore, mounted flush Kistler 9865B amplifiers were used. These amplifiers were connected to the nearby computer; thus, signals were directly displayed on the screen. These signals consist out of a vertical (FZ), cranio-caudal (FY) and medio-lateral (FX) direction. The signals were transferred into graphs and numbers after each measurement moment. The graphs and numbers of the cranio-caudal, vertical and vertical impulse were used for analysis. The walkway of the dog was surrounded by fence to guide the dog in the right direction. The handler walked or ran beside the animal, outside of the surrounding fence. If necessary, the handler vocally encouraged the dog. The walkway was 11 meters long and the force plate itself 40 cm long and 60 cm wide. The force plate can be adapted in size if needed, for most of the dogs a smaller plate was more suitable. These size-adaptations can be seen in FIG. 1 of Corbee et al. (2014, Vet. J. 202(1): p. 116-127).

Force plate analysis has been successfully used in dogs to evaluate lameness and/or the effects of treatment in a non-invasive, objective fashion (Kalis et al., 2012, Vet. Surg. 41(1): p. 148-155; Smolders et al., 2012, Vet. Surg. 41(6): p. 720-732; Van der Peijl et al., 2012, Vet. Comp. Orthop. Traumatol. 25(2): p. 126-134). After the publication of a study by McLaughlin et al. (1991, Vet. Surg. 20(5): p. 291-297), force plate analysis became an objective standard in the evaluation of canine locomotion. Force plate analysis has the benefit of measuring the propulsive force (FYmin), the braking force (FYmax), the vertical force (FZmax), the impulse (the vertical force*time, IZ) and the symmetry indices (SI) of them. Fy has been studied to evaluate the effects of lumbosacral decompressive surgery in dogs with degenerative lumbosacral stenosis (Van Klaveren et al., 2005, Vet. Surg. 34(5): p. 450-456). The propulsive force (FYmin) seems to be best correlated with canine brief pain inventory pain severity score in dogs with osteoarthritis (Brown et al., 2013, J. Veterinary Internal Medicine 27(1): p. 22-30). SI of FYmin was shown relevant in improvement of after surgical treatment of unilateral fragmented coronoid process in dogs (Theyse et al., 2000, Veterinary and Comparative Orthopaedics and Traumatology 13(3): p. 135-140). However, in this study, the SI seems less interesting to screen for OA dogs since there might be dogs with multiple lameness limbs. Therefore, in this study, FYmin was used as a parameter to categorize the lameness status of each paw, so also to label a dog as suffering osteoarthritis or not (healthy, or non-OA) at the start day (T1). First, data of forces of healthy dogs (Corbee et al., 2014, Vet. J. 202(1): p. 116-127) were exploited to establish the cut-off of healthy paws of each position. In this study, two SD (two times standard deviation or 2SD) from the mean was used as cut-off of FYmin force of each position of the paw, covering 95% of the data according to the three-sigma rule (Table 1).

TABLE 1
Cut-off of lameness paw (FYmin data of healthy dogs from
study by Corbee et al, 2014, Vet. J. 202(1): p. 116-127)

| Force | Paw | mean ± SD | +2SD | −2SD | +3SD | −3SD |
|---|---|---|---|---|---|---|
| FYmin | Left, Front (LF) | −1.06 ± 0.14 | −0.78 | −1.34 | −0.64 | −1.48 |
| FYmin | Left, Hind (LH) | −0.86 ± 0.11a | −0.64 | −1.08 | −0.53 | −1.19 |
| FYmin | Right, Front (RF) | −1.06 ± 0.09 | −0.88 | −1.24 | −0.79 | −1.33 |
| FYmin | Right, Hind (RH) | −0.87 ± 0.13a | −0.61 | −1.13 | −0.48 | −1.26 |

Then, FYmin of each paw of each dog in the trial was compared with the cut-off. Dogs with a paw performing a peak propulsive force (FYmin) smaller than the cut-off are categorized as OA dogs, otherwise as healthy dogs (i.e., non-OA dogs). Based on that classification, there were eight OA dogs, of which four received the supplementation with sialyllactose for 4 months (OA-SL, n=4) and four were fed standard diet (OA-placebo, n=4). The other four healthy dogs were equally divided into the SL-supplementation group (control-SL, n=2) or the placebo group (control-placebo, n=2).

Based on the value of FYmin of each paw at T1, an additional classification was proposed for lameness of the paws. Paws of OA dogs having FYmin value outside 2SD interval were classified as more lameness (ML) while paws of OA dogs with FYmin value within 2SD interval were assigned as less lameness (LL). Paws of healthy dogs (non-OA) are signified as healthy (or H). Hence, at individual paw level, there were three classes of paws: healthy, LL, and ML paws. To analyze the impact of SL supplementation on limb's forces (FYmin, FYmax, Fz, and IS), the statistical analysis was performed based on paws of the same classes of all dogs as percentage of changes at Tn (i.e., T2, T3 or T4) as compared to T1.

### Isolation of Peripheral Blood Mononuclear CD14+ Cells (CD14+ PBMCs), RNA Extraction and Transcriptomics (RNAseq)

Blood from each dog (vena jugularis) was collected in heparin tubes at start day (T1) and at two weeks (T2) after start day. From 20 ml of heparinized blood, peripheral blood mononuclear cells (PBMCs) were isolated via density gradient centrifugation using a standard protocol (Heinrich et al., 2017, PLoS One 12(8): e0183572). Briefly, blood samples were diluted 1:3 in phosphate buffered saline (PBS) containing 1% penicillin/streptomycin and added onto a gradient with equal volumes of histopaque 1.077 g/ml and 1.119 g/ml. After 30 minutes of centrifugation, cells were harvested using a Pasteur pipette, washed with PBS and 1% penicillin/streptomycin followed by depletion of erythrocyte contamination by hypotone lysis with distilled water. Subsequently, cells were resuspended in Roswell Park Memorial Institute (RPMI)-medium 1640 containing 10% fetal calf serum and 1% penicillin/streptomycin. Monocytes were isolated using the positive magnetic-sorting selection method (Qeska et al., 2013, Vet. Immunol. Immunopathol. 151(3-4): p. 181-192). First, PBMCs were incubated for 30 minutes at 4° C. with a mouse anti-canine CD14 (Abcam). Next, the suspension was washed with PBS, followed by incubating for 30 minutes at 4° C. with the secondary goat anti-mouse microbeads (Miltenyi Biotec) and then placed onto an MS column according to the manufacturer's instructions. Finally, after several washing steps, CD14⁺ cells were obtained, counted, and suspended in 1 ml of RNAprotect® cell reagent (QIAgen) for storage at −80° C.

Total RNA were extracted from CD14+ monocytes using RNeasy mini kit (QIAgen). First, 10⁶ cells were disrupted in the provided buffer and homogenized using QIAshredder (QIAgen). The homogenates were further manipulated according to the guidance of the manufacturer. The total purified RNA was eluted in 40 μl ultra-pure water and quickly checked for the concentration and quality using Nanodrop (Implen Nanophotometer®) before storage at −80° C.

Total RNA was submitted to Azenta (Genewiz, Germany) to perform RNA sequencing. The total RNA was checked again for quality and concentration using Qubit and fragment analyzer before the mRNA was selected for polyA library preparation and sequenced using the Illumina® Novaseq™ 2×150 bp technology platform guaranteed 20 million paired-end reads per samples (40 million reads). RNA sequencing reads were quality-checked with FastQC (version 0.11.9; Babraham Bioinformatics) and trimmed with Trimmomatic (version 0.40; USADeL LAB; Bolger et al., 2014, Bioinformatics 30(15): p. 2114-2120). Reads were aligned to the dog reference (CanFam3.1; NCBI) using the genome alignment method in STAR (Dobin et al., 2013, Bioinformatics 29(1): p. 15-21); Gene expression was quantified by featureCounts (Liao et al., 2014, Bioinformatics, 30(7): p. 923-930) and differential expression analysis was performed using DEseq2 (Love et al., 2014, Genome Biol. 15(12): 550, 21 pages). Enrichment analysis of differential expressed genes (DEGs) and ranked genes (based on −log 10(p-value)*sign(fold change)) was done in clusterProfiler (Wu et al., 2021, Innovation (Camb) 2(3): 100141) using the Hallmark geneset (igordot.github.io/msigdbr/).

### Statistical Analysis

Data statistics of clinical and metagenomics data and figure plotting were performed in RStudio (rstudio.com). Clinical data was analyzed using the Wilcoxon paired test for comparing each timepoint versus T1 for each group. The percentage change versus T1 for each timepoint was used as input to compare the treatments using the Mann-Whitney U test.

### Results

In terms of safety, there were no adverse effects from the supplementation with 3′sialyllactose and 6′sialyllactose as registered by the research team at the Utrecht University Faculty of Veterinary Medicine.

After 2 (T3) and 4 months (T4) of treatment, sialyllactose strengthened FYmin of healthy paws of non-OA dogs (P=0.083). In the LL class (non-lameness paws of OA dogs), there was no significant difference in changes of FYmin between SL treatment and the placebo (P=0.878 and 0.645, respectively). Interestingly, FYmin were improved with 15% in ML paws (lameness paws of OA-dogs) of dogs with sialyllactose treatment after 4 months (T4) while there was no improvement in the paws of the placebo dogs (P=0.015). FIG. 1 exhibits FYmin changes over time in all paw classes.

Like FYmin, changes over time of FYmax of the LL paws were not different between sialyllactose treatment and the placebo. Remarkably, treatment with sialyllactose enhanced FYmax of healthy paws at all timepoints while there were slightly changes in the placebo as compared to the start day. At T4, such changes of FYmax in sialyllactose treated group were significantly higher than in the placebo (P<0.01). However, in the ML paws class, the changes over time of FYmax were barely seen although such changes were slightly higher but not significant, in sialyllactose treatment than in the placebo, especially at T4 (P=0.161). FIG. 2 reveals FYmax changes over time in all paw classes.

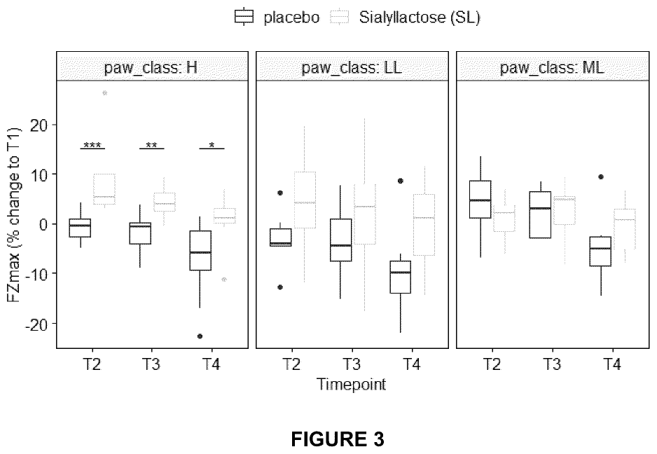

Changes in FZmax were narrower than changes in FYmin and FYmax, ranging 5-10% as compared to T1. The changes in FZmax tended to be worse in the placebo but better in the sialyllactose treated group for all paw classes. There is a trend of improving FZmax of LL paw class in the sialyllactose group versus placebo at T4 (P=0.065). However, the differences between changes in the sialyllactose treated group and the placebo were not significant in the ML paw class (P=0.195). In contrast, FZmax changes in the healthy paws with sialyllactose treatment were significantly higher than such changes in the same paw class of the placebo at T2 (P<0.001), T3 (P<0.01) and T4 (P<0.05). FIG. 3 shows FZmax changes over time in all paw classes.

Like FZmax, changes during trial in the impulse (IZ) were limited in all paw classes. There was no significant difference between changes of IZ of the sialyllactose treated group and the placebo.

In general, OA is an inflammatory disease of the joint so that it has impact on the force performance of dogs. The disease lasts for long term with pain peak up and down, hence forces can change over time. Force performance of paws can be used to study the lameness status of dogs. Among the forces, the propulsive force (FYmin) is suitable to screen, categorize or classify lameness paws. SLs supplementation improved 15% propulsive force of severe lameness dogs while such change was not seen in the placebo. In addition, SLs treatment also improved the breaking force and the vertical force of the healthy paws. Changes in the impulse and the symmetry of all forces were limited and there was no difference between treatment and the placebo.

### Sialyllactose Promotes Polarization of CD14+ PBMCs Toward Less Inflammatory Status

Gene expression of CD14+ PBMCs at T2 (2 weeks after start day) was quantified by featureCounts and differential expression analysis was performed using DEseq2 to compare the expression profile of SL-treated dogs' CD14+ cells versus the ones of the placebos. Following differential expression analysis and ranked genes (based on −log 10(p-value)*sign(fold change)) was done using the Hallmark geneset (igordot.github.io/msigdbr). Genes normally act in a whole set. A hallmark consists of a refined gene set, that conveys a specific biological state or process and displays coherent expression (Liberzon et al., 2015, Cell. Syst. 1(6): p. 417-425). Table 2 summarizes differential expression in hallmarks between sialyllactose treatment versus the placebo in the healthy and the OA groups at T2.

TABLE 2
Hallmarks of significantly differential expression in peripheral blood mononuclear
CD14⁺ cells of the control and osteoarthritis dogs treated with sialyllactose versus placebo
at 2 weeks after start (T2). NES: normalized enrichment score.

| Control (non-OA) dogs (SLs versus placebo) | | Osteoarthritis dogs (SLs versus placebo) | |
|---|---|---|---|
| Hallmarks | NES | Hallmarks | NES |
| TNFA SIGNALING VIA NFKB | −1.46 | IL6 JAK STAT3 SIGNALING | −1.79 |
| | | TNFA SIGNALING VIA NFKB | −2.01 |

The most differential expressed gene in the dataset is DUSP2 (Dual Specificity Phosphatase 2; p-value: 0.000019 and log 2FC=−1.08 for OA dogs), which is a marker gene in the HALLMARK_TNFA_SIGNALING_VIA_NFKB geneset. DUSP2 has a binding site for NF-κB in the promoter (Kanemaru et al. 2018, Int Immunol. 24; 30(6): p. 255-265). The protein encoded by this gene is a member of the dual specificity protein phosphatase subfamily. DUSP2 is expressed at high levels in immune cells, particularly in patients with inflammatory arthritis, including rheumatoid arthritis, and DUSP2-knockout mice present with a significant reduction in inflammatory responses (Hamamura et al., 2015, Cell Signal. 27(4): p. 828-35).

In OA dogs, sialyllactose supplementation down-regulated genes related to the hallmarks IL6-JAK-STAT3 signaling and TNFα signaling via NF-κ-β, which are known as inflammatory associated hallmarks. TNFα together with IL-6 signaling expression in CD14⁺ cells indicate a promotion of inflammatory monocytes (Loukov et al., 2018, Osteoarthritis Cartilage 26(2): p. 255-263; Shi et al., 2019, Biomed. Res. Int.: 4291689). The fact that the inflammation hallmarks were downregulated in monocytes and limb's forces were recuperated upon sialyllactose supplementation indicates sialyllactose modulate the immune system to alleviate the chronic inflammatory disease.

Like in OA dogs' monocytes, supplementation of sialyllactose in the control (non-OA) dogs also reduced the inflammatory associated pathway TNFα signaling via NF-κ-β as compared to the placebo dogs. Nevertheless, there was no difference in the hallmarks IL6-JAK-STAT3 signaling between sialyllactose-treated and the placebos, indicating there might be less inflammation in the control dogs as compared to the OA ones. In conclusion, sialyllactose directly modulates monocytes circulating in the blood to improve joint health. Sialyllactose supplementation steered the fate of the systemic monocytes toward less inflammatory and therefore improved the limb's forces of the dogs.

1.-23. (canceled)

24. A method of polarizing peripheral blood mononuclear CD14+ cells to a less inflammatory status in a subject diagnosed with or that is at risk of developing an inflammatory disease or autoimmune disease, the method comprising:
administering a sialylated saccharide to the subject in an amount sufficient to polarize the peripheral blood mononuclear CD14+ cells in the subject to the less inflammatory status.

25. The method according to claim 24, wherein the disease is a monocyte-driven disease.

26. The method according to claim 24, wherein the inflammatory disease is a chronic inflammatory disease.

27. The method according to claim 24, wherein the subject is diagnosed or at risk of developing arthritis.

28. The method according to claim 27, wherein the subject is diagnosed with or at risk of developing osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia, and/or enthesitis-related arthritis.

29. The method according to claim 24, wherein the subject is diagnosed or at risk of developing osteoarthritis.

30. The method according to claim 24, wherein the sialylated saccharide comprises:
(i) a sialic acid that is linked to a monosaccharide in an α-2,3- or α-2,6-linkage and wherein the monosaccharide is galactose or N-acetylglucosamine; and
(ii) a lactose, a lacto-N-biose (LNB) or N-acetyllactosamine (LacNAc) at its reducing end.

31. The method according to claim 30, wherein the sialylated saccharide is a milk oligosaccharide.

32. The method according to claim 30, wherein the sialylated saccharide consists of 3-9 monosaccharides.

33. The method according to claim 30, wherein the sialylated saccharide is selected from the group consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc), and sialyl Lewis x.

34. A method of polarizing peripheral blood mononuclear CD14+ cells to a less inflammatory status in a subject diagnosed or that is at risk of developing an inflammatory disease or autoimmune disease, the method comprising:
administering a first sialylated saccharide and a second sialylated saccharide to the subject in amounts sufficient to polarize the peripheral blood mononuclear CD14+ cells in the subject to the less inflammatory status.

35. The method according to claim 34, wherein the disease is a monocyte-driven disease.

36. The method according to claim 34, wherein the inflammatory disease is a chronic inflammatory disease.

37. The method according to claim 34, wherein the subject is diagnosed or at risk of developing arthritis.

38. The method according to claim 37, wherein the subject is diagnosed with osteoarthritis, rheumatoid arthritis, psoriatic arthritis, juvenile idiopathic arthritis, ankylosing spondylitis, systemic lupus erythematosus, gout, enteropathic arthritis, juvenile arthritis (i.e., childhood arthritis), reactive arthritis, drug-induced lupus, fibromyalgia, and/or enthesitis-related arthritis.

39. The method according to claim 34, wherein the subject is diagnosed with osteoarthritis.

40. The method according to claim 34, wherein the first sialylated saccharide comprises:
(i) a sialic acid that is linked to a monosaccharide in an α-2,3-linkage and wherein the monosaccharide is galactose or N-acetylglucosamine; and
(ii) a lactose, a lacto-N-biose (LNB) or N-acetyllactosamine (LacNAc) at its reducing end.

41. The method according to claim 40, wherein the first sialylated saccharide is selected from the list consisting of 3′-sialyllactose (3′SL), 3,6-disialyllactose, 8,3-disialyllactose, 3'S-2′FL, 3'S-3-FL, LST a, LST d, 3′-sialyllacto-N-biose (3′SLNB), sialyl Lewis a, 3′-sialyllactosamine (3′SLacNAc), and sialyl Lewis x.

42. The method according to claim 40, wherein the first sialylated saccharide is 3′-sialyllactose (3′SL).

43. The method according to claim 34, wherein the second sialylated saccharide comprises:
(i) a sialic acid that is linked to a monosaccharide in an α-2,6-linkage and wherein the monosaccharide is galactose or N-acetylglucosamine; and
(ii) a lactose, a lacto-N-biose (LNB) or N-acetyllactosamine (LacNAc) at its reducing end.

44. The method according to claim 43, wherein the second sialylated saccharide is selected from the list consisting of 6′-sialyllactose (6′SL), 3,6-disialyllactose, 6,6-disialyllactose, 6'S-2′FL, 6'S-3-FL, LST b, LST c, 6′-sialyllacto-N-biose (6′SLNB), and 6′-sialyllactosamine (6′SLacNAc).

45. The method according to claim 43, wherein the second sialylated saccharide is 6′-sialyllactose (6′SL).

46. The method according to claim 34, wherein the amount of the first sialylated saccharide administered to the subject is 0.4 to 50.0 times the amount of the second sialylated saccharide administered to the subject.

47. The method according to claim 34, wherein the first sialylated saccharide and the second sialylated saccharide are administered as part of a composition.
